(19) United States
(12) Patent Application Publication
(10) Pub. No.: US 2024/0091390 A1
(43) Pub. Date: Mar. 21, 2024

(54) IMPROVED CHOLECYSTOKININ-2 RECEPTOR (CCK2R) TARGETING FOR DIAGNOSIS AND THERAPY

(71) Applicant: Medizinische Universität Innsbruck, Innsbruck (AT)

(72) Inventors: Elisabeth VON GUGGENBERG ZU RIEDHOFEN, Innsbruck-Igls (AT); Anton Amadeus HÖRMANN, Innsbruck (AT)

(21) Appl. No.: 18/263,797

(22) PCT Filed: Feb. 2, 2022
(86) PCT No.: PCT/EP2022/052459

§ 371 Date: Aug. 1, 2023

Related U.S. Application Data
(63) Continuation-in-part of application No. PCT/EP2021/052428, filed on Feb. 2, 2021.

(51) Int. Cl. A61K 51/08; A61P 35/00; C07K 7/02

(52) U.S. Cl. CPC A61K 51/088; A61P 35/00; C07K 7/02; A61K 2121/00; A61K 2123/00

(57) ABSTRACT

The present invention provides valuable peptidomimetics for therapeutic and diagnostic purposes as well as compositions, methods, uses and kits based on these peptidomimetics. In particular, the peptidomimetics of the present invention are incorporated by CCK2R expressing cells, for instance, cancer cells. This allows, for instance, to selectively destroy cancer cells or to selectively image cancer cells that express CCK2R.

### BACKGROUND OF THE INVENTION

The invention among other things relates to a peptidomimetic with improved properties for specific cholecystokinin-2 receptor (CCK2R) targeting and its diagnostic and therapeutic use. Cholecystokinin receptors are classified into two receptor subtypes, CCK1R and CCK2R. CCK2R have been identified in various tumours such as neuroendocrine tumours, medullary thyroid carcinomas (MTC), small cell lung cancers (SCLC), leiomyosarcomas/leiomyomas, gastrointestinal stromal tumours, insulinomas, vipomas, carcinoids, astrocytomas, stromal ovarian cancers, breast and endometrial adenocarcinomas, and others (Reubi J C et al., Cancer Res 1997, 57: 1377-1386; Reubi J C, Curr Top Med Chem 2007, 7: 1239-1242; Sanchez C et al. Mol Cell Endocrinol 2012, 349: 170-179), while CCK1R are expressed only in a limited number of human tumours. Different radiolabelled peptide probes have been developed based on the endogenous ligands for CCK2R, cholecystokinin (CCK) or gastrin. The two peptides CCK and gastrin bind to CCK2R with almost the same affinity and potency and share a common bioactive region at the C-terminus, Trp-Met-Asp-Phe (Dufresne M et al., Physiol Rev 2006, 86: 805-847), which proved to be essential for receptor binding (Tracy H J et al., Nature 1964, 204: 935-938). Due to the very short physiological half-life of the parent peptide, additional synthetic modifications of the peptide are generally required to metabolically stabilize the linear amino acid sequence for medical applications (Fani M et al., Theranostics 2012, 2: 481-501). Such modifications have been explored also for radiolabelled CCK and gastrin analogues (Roosenburg S et al., Amino Acids 2011, 41: 1049-1058). Beside radioligands based on Asp-Tyr-Met-Gly-Trp-Met-Asp-Phe-NH₂ (CCK8) and Leu-Glu-Glu-Glu-Glu-Glu-Ala-Tyr-Gly-Trp-Met-Asp-Phe-NH₂ (minigastrin, MG), also nonpeptidic ligands have been proposed (Wayua C et al., J Nucl Med 2015, 56: 113-119). CCK2R specific tumour uptake could be recently proven also in nude mice bearing tumour xenografts derived from colorectal adenocarcinoma cells by optical imaging with a fluorescent CCK2R-targeted MG analogue (dQ-MG-754) (Kossatz S et al., Biomaterials 2013, 34: 5172-5180). By using the potent CCK2R ligand Z-360 conjugated to tubulysin B delivering the cytotoxic drug selectively to CCK2R-positive tumours tumour regression could be observed in a preclinical animal model (Wayua C et al., Mol Pharm 2015, 12: 2477-2483).

Using radiolabelled [diethylenetriaminepentaacetic⁰,D-Glu¹]minigastrin (DTPA-MG0), good tumour targeting properties have been obtained, however, also a very high kidney uptake was observed leading to severe renal side effects in patients treated with ⁹⁰Y-labelled DTPA-MG0 (Béhé M et al., Biopolymers 2002, 66: 399-418). ¹¹¹In-DTPA-MG0 has however proven to be superior to somatostatin receptor scintigraphy and 2-deoxy-2-[¹⁸F]fluoro-D-glucose positron emission tomography (FDG PET) for tumour detection in patients with MTC and SCLC and has shown to be of additional value also in neuroendocrine tumours with low somatostatin receptor expression (Gotthardt M et al., Eur J Nucl Med Mol Imaging 2006, 33: 1273-1279; Gotthardt M et al., Endocr Relat Cancer 2006, 13: 1203-1211).

With the truncated ¹¹¹In-labelled peptide analogue, [1,4,7,10-tetraazacyclodocecan-1,4,7,10-tetraacetic acid⁰,D-Glu¹,DesGlu²⁻⁶]minigastrin (DOTA-MG11) a significantly reduced kidney uptake was shown in animal studies (Behe M et al., Eur J Nucl Med Mol Imaging 2005: 32, S78) and confirmed in a first clinical study (Fröberg A C et al., Eur J Nucl Med Mol Imaging 2009, 36: 1265-1272). However, due to a clearly reduced stability against enzymatic degradation and low biological half-life below five minutes in vivo (Breeman W A et al., Nucl Med Biol 2008, 35: 839-849) only a poor diagnostic efficacy was observed with ¹¹¹In-DOTA-MG11. To increase enzymatic stability and improve specific receptor targeting while reducing kidney retention different modifications in the peptide sequence have been attempted in the N-terminal region of the peptide (Aloj Let al., Eur J Nucl Med Mol Imaging 2011, 38: 1417-1425; Laverman P et al., Eur J Nucl Med Mol Imaging 2011, 38: 1410-1416). Only very view modifications have been attempted also in the C-terminal region and are limited to substitution of methionine with unnatural amino acids such as norleucine or homopropargylglycin to prevent methionine oxidation associated with loss of receptor affinity (Mather S J et al., J Nucl Med 2007, 48: 615-622; Roosenburg S et al., Bioconjug Chem 2010, 21: 663-670). However, the analysis of blood and urine of BALB/c mice injected with these different new peptide analogues labelled with ¹⁷⁷Lu, has shown, that already 10 min after injection no intact radioligand could be detected (Ocak M et al., Eur J Nucl Med Mol Imaging 2011, 38: 1426-1435). Also the non-canonical amino acid methoxinine has been recently used to substitute the oxidation sensitive methionine residue and chemically stabilize MG analogues (Grob N M et al., J Pept Sci 2017, 23: 38-44), without improving however the biological half-life. The above mentioned studies make clear that the chemical modifications adopted so far are not successful in improving the biological half-life and targeting properties in vivo.

The present inventors have previously generated two ¹¹¹In-labelled peptide analogues with the structures ¹¹¹In-DOTA-DGIu-Ala-Tyr-Gly-Trp-Met-Asp-1Nal-NH₂ and ¹¹¹In-DOTA-DGIu-Ala-Tyr-Gly-Trp-(N-Me)Nle-Asp-(NMe)Phe-NH₂, referred to as ¹¹¹In-DOTA-MGS1 and ¹¹¹In-DOTA-MGS4, respectively (Klingler M et al., Eur J Nucl Med Mol Imaging 2017, 44: S228). In addition, the present inventors previously developed DOTA-DGIu-Ala-Tyr-Gly-Trp-(NMe)Nle-Asp-1Nal-NH₂ (WO 2018/224665 A1), referred to DOTA-MGS5, which showed good stability and cell internalization properties.

Based on further developments, the present inventors surprisingly found that a new group of peptidomimetics that target CCK2R possesses superior properties, in particular an improved biodistribution and stability, which makes the peptidomimetics of the present invention particularly suitable for imaging and therapeutic applications.

### SUMMARY OF THE INVENTION

It is an objective of the present invention to improve the stability, e.g. half-life in serum, and biodistribution profile of CCK2R targeting peptidomimetics. In particular, the invention reduces kidney retention of the peptidomimetics and therefore avoids or reduces renal toxicity. This objective is achieved according to the present invention by the following embodiments and aspects of the invention.

In one aspect, the invention provides a peptidomimetic of the structure

X-Linker-βAla-Trp-(NMe)Nle-Asp-1Nal,

wherein X is a chelator comprising a radionuclide or a prosthetic group comprising a radionuclide.

In some embodiments the chelator chelates the radionuclide. In some embodiments the radionuclide is covalently bound to the prosthetic group.

In some embodiments the radionuclide is selected from the group consisting of ²²⁵Ab, ²¹²Bi, ²¹³Bi, ⁶²Cu, ⁶⁷Cu, ⁶⁹Cu, ⁶⁶Ga, ⁶⁷Ga, ⁶⁸Ga, ¹¹¹In, ^{113m}In, ¹⁷⁷Lu, ¹⁸⁶Re, ¹⁸⁸Re, ⁴³Sc, ⁴⁴Sc, ⁴⁷Sc, ¹⁵⁵Tb, ¹⁶¹Tb, ^{99m}Tc, ⁸⁶Y, ⁹⁰Y, ¹⁶⁹Yb, ¹⁷⁵Yb, ⁵²Fe, ¹⁶⁹Er, ⁷²As, ⁹⁷Ru, ²⁰³Pb, ²¹²Pb, ⁵¹Cr, ^{52m}Mn, ⁸⁹Zr, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ¹⁴²Pr, ¹⁴³Pr, ¹⁹⁸Au, ¹⁹⁹Au, ¹²³I, ¹²⁴I, ¹²⁵I, Al¹⁸F and ¹⁸F.

In some embodiments of the invention, the radionuclide is a halogen (radiohalide). In particular, the radionuclide may be a halogen when X is a prosthetic group and the radiohalide is covalently bound to the prosthetic group to couple the radiohalide to the peptide sequence βAla-Trp-(NMe)Nle-Asp-1Nal via the Linker.

In some embodiments the chelator is

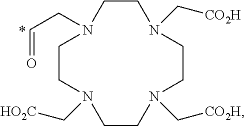 [SMILES: O=CCN1CCN(CC(=O)O)CCN(CC(=O)O)CCN(CC(=O)O)CC1]

wherein the asterisk indicates the position where the chelator is directly bound to the Linker. For instance, the marked carbonyl carbon may be directly bound to a nitrogen of the Linker forming an amide bond with the Linker.

In a preferred embodiment the peptidomimetic has the structure DOTA-Linker-βAla-Trp-(NMe)Nle-Asp-1Nal.

In some embodiments the Linker is selected from the group consisting of GABA-GABA, GABOB-GABOB or γDGlu-γDGlu.

In some embodiments the radionuclide is selected from the group consisting of Al¹⁸F, ²²⁵Ac, ²¹²Bi, ²¹³Bi, ⁶²Cu, ⁶⁴Cu, ⁶⁷Cu, ⁶⁹Cu, ⁶⁶Ga, ⁶⁷Ga, ⁶⁸Ga, ¹¹¹In, ¹¹³min, ¹⁷⁷Lu, ¹⁸⁶Re, ¹⁸⁸Re, ⁴³Sc, ⁴⁴Sc, ⁴⁷Sc, ¹⁵⁵Tb, ¹⁶¹Tb, ^{99m}Tc, ⁸⁶Y, ⁹⁰Y, ¹⁶⁹Yb, and ¹⁷⁵Yb, in particular when the peptidomimetic comprises a chelator and the radionuclide is chelated by the chelator. Preferred radionuclides to be chelated by the chelator, such as DOTA, are ⁹⁰Y, ¹¹¹In, ⁶⁸Ga or ¹⁷⁷Lu. Al¹⁸F can be chelated by DOTA.

Preferably, the peptidomimetic comprises 5 to 10 amino acids. For instance, the peptidomimetic may comprise 5, 6, 7, 8, 9 or 10 amino acids. DOTA as used herein refers to 1,4,7,10-Tetraazacyclododecane-1,4,7,10-tetraacetic acid. In a preferred embodiment, the peptidomimetic is DOTA-GABOB-GABOB-βAla-Trp-(NMe)Nle-Asp-1Nal, wherein GABOB refers to γ-amino-β-hydroxybutyric acid. It is preferred that the peptidomimetic comprises a radionuclide that is chelated by DOTA.

The peptidomimetic of the invention specifically binds to CCK2R.

The Linker of the invention connects the amino acid polymer βAla-Trp-(NMe)Nle-Asp-1Nal with DOTA. In some embodiments, the Linker does not reduce or substantially reduce the binding affinity of the amino acid polymer βAla-Trp-(NMe)Nle-Asp-1Nal to CCK2R.

In some embodiments the radionuclide of the peptidomimetic of the invention is selected from the group consisting of ²²⁵Ac, ²¹²Bi, ²¹³Bi, ⁶²Cu, ⁶⁴Cu, ⁶⁷Cu, ⁶⁹Cu, ⁶⁶Ga, ⁶⁷Ga, ⁶⁸Ga, ¹¹¹In, ^{113m}In, ¹⁷⁷Lu, ¹⁸⁶Re, ¹⁸⁸Re, ⁴³Sc, ⁴⁴Sc, ⁴⁷Sc, ¹⁵⁵Tb, ¹⁶¹Tb, ^{99m}Tc, ⁸⁶Y, ⁹⁰Y, ¹⁶⁹Yb, and ¹⁷⁵Yb. Preferably the radionuclide is ⁹⁰Y, ¹¹¹In, ⁶⁸Ga or ¹⁷⁷Lu. Other radionuclides that are chelated by the chelator, such as DOTA, may also be chosen by the skilled person.

Preferably, the C-terminus of the peptidomimetic of the invention is amidated.

In some embodiments the peptidomimetic is ⁶⁸Ga-DOTA-GABOB-GABOB-βAla-Trp-(NMe)Nle-Asp-1Nal-NH₂ (⁶⁸Ga-DOTA-MGSA). In further embodiments the peptidomimetic is ¹⁷⁷Lu-DOTA-GABOB-GABOB-βAla-Trp-(NMe)Nle-Asp-1Nal-NH₂ (¹⁷⁷Lu-DOTA-MGSA).

In some embodiments, the peptidomimetic is ⁶⁸Ga-DOTA-γDGlu-γDGlu-βAla-Trp-(NMe)Nle-Asp-1Nal-NH₂ or ¹⁷⁷Lu-DOTA-γDGlu-γDGlu-βAla-Trp-(NMe)Nle-Asp-1Nal-NH₂.

The peptidomimetic DOTA-MGSA, in particular ⁶⁸Ga-DOTA-MGSA and ¹⁷⁷Lu-DOTA-MGSA are preferred embodiments of the invention. In particular, it is preferred to carry out the different methods and uses of the present invention with ⁶⁸Ga-DOTA-MGSA or ¹⁷⁷Lu-DOTA-MGSA.

Preferably, the peptidomimetic comprises 5 to 10 amino acids. Most preferably, the peptidomimetic comprises 7 amino acids. In some embodiments the 7 amino acids are GABOB-GABOB-βAla-Trp-(NMe)Nle-Asp-1Nal.

In another aspect, the present invention provides a method of producing the peptidomimetic of the present invention comprising synthesizing the peptidomimetic. In some embodiments, the method of producing the peptidomimetic comprises solid phase peptide synthesis as known to the skilled person.

In one aspect of the invention, the invention provides a pharmaceutical composition comprising the peptidomimetic of the present invention and a pharmaceutically acceptable carrier. The pharmaceutical composition of the invention may be used for therapeutic or diagnostic purposes. Depending on the intended use, the skilled person can choose the pharmaceutically acceptable carrier.

In a further aspect of the invention, the invention provides a use of the peptidomimetic of the invention for imaging a tumour. In some embodiments the tumour to be imaged expresses CCK2R on the surface of the tumour cells.

In a further aspect of the invention, the invention provides a method of imaging cancer cells, wherein the method comprises the steps of a) contacting a cancer cell with the peptidomimetic of the invention, thereby bringing the radionuclide in contact with the cancer cell, and b) visualizing the radionuclide that is in contact with the cancer cell. In some embodiments the step of contacting the cancer cell with the peptidomimetic of the invention comprises administering the peptidomimetic to a patient. In some embodiments the patient suffers from cancer. In some embodiments the cancer expresses CCK2R on the surface of the cancer cell. Preferably, the method of imaging uses the peptidomimetics ⁶⁸Ga-DOTA-GABOB-GABOB-βAla-Trp-(NMe)Nle-Asp-1Nal-NH₂ or ¹⁷⁷Lu-DOTA-GABOB-GABOB-βAla-Trp-(NMe)Nle-Asp-1Nal-NH₂.

The cancer to be treated, diagnosed or imaged in accordance with the technical teaching of the present invention can be selected from the cancers disclosed elsewhere in the present application.

In one aspect of the invention, the invention provides the peptidomimetic of the invention for use in the treatment of a cancer. Preferably, the cancer expresses CCK2R on the surface of cancer cells.

In a further aspect of the invention, the invention provides the peptidomimetic of the invention for use in diagnosing cancer. Preferably, the cancer expresses CCK2R on the surface of cancer cells.

In a further aspect of the invention, the invention provides a method of treating a patient suffering from a disease, the method comprising administering to the patient the peptidomimetic of the invention. Preferably, the disease is a cancer and more preferably the cancer expresses CCK2R on the surface of cancer cells.

In a further aspect of the invention, the invention provides a method of diagnosing cancer in a patient, wherein the method comprises the steps of a) contacting a cancer cell of the patient with the peptidomimetic of the invention, thereby bringing the radionuclide in contact with the cancer cell, and b) visualizing the radionuclide that is in contact with the cancer cell. In some embodiments, the cancer to be diagnosed expresses CCK2R on the surface of cancer cells.

In preferred embodiments, the peptidomimetic of the present invention specifically binds to CCK2R. Specific binding to CCK2R allows targeting of cells and tissue that express CCK2R over cells and tissue that does not express CCK2R. This characteristic of the peptidomimetic of the present invention is useful for diagnostic and therapeutic purposes, for example, for the diagnosis and treatment of certain types of cancer, in particular cancers that express CCK2R. The teaching of the present invention is, however, not limited to cancer, but pertains to any disease that is associated with the expression of CCK2R. In particular, the peptidomimetic of the present invention is useful for diagnostic and therapeutic purposes, since it shows a high level of cellular uptake (cellular internalization) by cells that express CCK2R. Further, the peptidomimetic of the present invention is useful, since it is particularly stable against degradation, in particular degradation in serum by proteases, preferably metabolic degradation in vivo by a variety of proteolytic enzymes. Moreover, the peptidomimetic of the present invention is useful, since it does not accumulate in the kidneys, or only accumulates in the kidneys to a low level that is not harmful to the patient that is treated or diagnosed with the peptidomimetic of the present invention.

It is emphasized that any combination of the aspects and embodiments disclosed herein, or the respective technical features, is also disclosed herein as part of the present invention. The skilled person understands that the present invention is not limited to the embodiments explicitly recited above, but also includes combinations not explicitly disclosed above or below. Such combinations result, for example, from combining aspects, embodiments or features of the present invention mentioned above with aspects, embodiments or features of the present invention disclosed below in the detailed description of the present invention. The skilled person will appreciate that, in fact, the above described aspects and embodiments are to be considered together with the corresponding sections of the detailed description given further below, as well as with the examples set out in this application. Further, any headings given in the description are not to be construed as being limiting on the subject matter disclosed under the heading, that is, the subject matter disclosed under one heading can be read in combination with the subject matter described under a different heading.

Other aspects, embodiments, features, and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from the detailed description.

### BRIEF DESCRIPTION OF DRAWINGS

The invention will be described in more detail in the following section and illustrated in accompanying figures showing in:

FIG. 1: Cell internalization of A) the ⁶⁸Ga-labelled DOTA peptidomimetic variants ⁶⁸Ga-DOTA-MGSA (MGSA), ⁶⁸Ga-DOTA-MGSB (MGSB), and ⁶⁸Ga-DOTA-MGSC (MGSC) after 2 h incubation on A431-CCK2R and A431-mock cells as well as of B) ¹⁷⁷Lu-labelled DOTA-MGSA in A431-CCK2R/mock cells and AR42J cells (including blocking).

FIG. 2: Biodistribution in A431-CCK2R and A431-mock tumour-xenograft bearing nude mice at 4 h p.i. of the ¹⁷⁷Lu-labelled peptidomimetic DOTA-MGSA of the present invention in comparison with ¹⁷⁷Lu-DOTA-MGS5, Values are expressed as percentage of injected activity per gram (% IA/g; mean±SD, n=5).

FIG. 3: Biodistribution in A431-CCK2R and A431-mock tumour-xenograft bearing nude mice at 4 h p.i. of the ¹⁷⁷Lu-labelled peptidomimetic DOTA-MGSA of the present invention in comparison with ¹⁷⁷Lu-DOTA-MGS5 and co-injected with an excess of unlabelled peptide, Values are expressed as percentage of injected activity per gram (% IA/g; n=1).

FIG. 4: Biodistribution in A431-CCK2R and A431-mock tumour-xenograft bearing nude mice at 4 h p.i. of ¹⁷⁷Lu-DOTA-MGSA in comparison with other ¹⁷⁷Lu-labelled peptidomimetics. Values are expressed as percentage of injected activity per gram (% IA/g; mean±SD, n=5 for DOTA-MGSA and DOTA-MGS5; n=4 for the other peptidomimetics).

FIG. 5: Tumour-to-kidney ratio obtained for different ¹⁷⁷Lu-labelled peptidomimetics from biodistribution studies in A431-CCK2R and A431-mock tumour-xenograft bearing nude mice at 4 h p.i. (n=5 for DOTA-MGSA and DOTA-MGS5; n=4 for the other peptidomimetics).

FIG. 6: Biodistribution in A431-CCK2R tumour-xenograft bearing nude mice up to 7 days p.i. for A) ¹⁷⁷Lu-DOTA-MGS5 and B) ¹⁷⁷Lu-DOTA-MGSA. Values are expressed as percentage of injected activity per gram (% IA/g; mean±SD, n=5 for DOTA-MGSA and DOTA-MGS5).

FIG. 7: Retention of the peptidomimetics ¹⁷⁷Lu-DOTA-MGSA and ¹⁷⁷Lu-DOTA-MGS5 in different tissues over time: A431-CCK2R tumour-xenograft, blood, kidney, liver, pancreas, stomach. Values are expressed as percentage of injected activity per gram (% IA/g; mean±SD, n=5).

FIG. 8: Biodistribution in A431-CCK2R tumour-xenograft bearing nude mice at 1 h p.i. of ⁶⁸Ga-DOTA-MGSA and ⁶⁸Ga-DOTA-MGSC in comparison with ⁶⁸Ga-DOTA-MGS5. Values are expressed as percentage of injected activity per gram (% IA/g; mean±SD, n=3 for DOTA-MGSA and DOTA-MGSC, n=4 for DOTA-MGS5).

FIG. 9: Tumour-to-kidney ratio obtained for different ⁶⁸Ga-labelled peptidomimetics from biodistribution studies in A431-CCK2R tumour-xenograft bearing nude mice at 1 h p.i. (n=3 for DOTA-MGSA and DOTA-MGSC and n=4 for DOTA-MGS5).

FIG. 10: Small animal μPET/CT of A431-CCK2R tumour-xenograft bearing nude mice injected with ⁶⁸Ga-DOTA-MGS5 and ⁶⁸Ga-DOTA-MGSC at 1 h p.i. (scale ranging from 0.02 to 0.2 MBq/ml; CT in gray scale).

FIG. 11: Stability against enzymatic degradation in vivo as analysed by radio-HPLC of a blood sample taken from BALB/c mice after intravenous injection of the ¹⁷⁷Lu-labelled peptidomimetic DOTA-MGSA of the present invention in comparison with other ¹⁷⁷Lu-labelled peptides at 30 min p.i.: radiochemical purity after radiolabelling (dotted line), radio-HPLC of the blood sample (solid line).

FIG. 12: Stability against enzymatic degradation in vivo as analysed by radio-HPLC of a blood sample taken from BALB/c mice after intravenous injection of the ⁶⁸Ga-labelled peptidomimetics DOTA-MGSA, DOTA-MGSB and DOTA-MGSC of the present invention at 10 min p.i.: radiochemical purity after radiolabelling (dotted line), radio-HPLC of the blood sample (solid line).

### DETAILED DESCRIPTION OF THE INVENTION

All publications, including but not limited to patents, patent applications and scientific publications, cited in this description are herein incorporated by reference for all purposes as if each individual publication were specifically and individually indicated to be incorporated by reference.

The use of the term “comprising” as well as other grammatical forms such as “comprises” and “comprised” is not limiting. The terms “comprising”, “comprises” and “comprised” should be understood as referring to an open-ended description of an embodiment of the present invention that may, but does not have to, include additional technical features in addition to the explicitly stated technical features. In the same sense, the term “involving” as well as other respective grammatical forms such as “involves” and “involved” is not limiting. The same applies for the term “including” and other grammatical forms such as “includes” and “included”. Section headings throughout the description are for organizational purposes only. In particular, they are not intended as limiting for various embodiments described therein, and it is to be understood that embodiments (and features therein) described under one subheading may be freely combined with embodiments (and features therein) described under another subheading. Further, the terms “comprising”, “involving” and “including”, and any grammatical forms thereof, are not to be interpreted to exclusively refer to embodiments that include additional features to those explicitly recited. These terms equally refer to embodiments that consist of only those features that are explicitly mentioned.

As used herein, the term “peptidomimetic”, “peptide analogue” or “peptide derivative” or “peptide conjugate” refers to a compound that comprises a polymer of two or more amino acids that comprises at least one unnatural amino acid, pseudopeptide bond or chemical moiety that is different from an amino acid, such as a reporter group or a cytotoxic group, including a chelator, a prosthetic group, a Linker or a pharmacokinetic modifier. In particular, the peptidomimetics of the invention comprise group X, wherein X is a chelator comprising a radionuclide or a prosthetic group comprising a radionuclide, and a Linker. A peptidomimetic as defined herein generally mimics a biological activity of a natural peptide. In the present case, the peptidomimetic of the present invention mimics the ability, in the sense of having the ability, of natural CCK2R ligands, such as gastrin, to specifically bind to CCK2R.

As used herein, the term “amino acid polymer” refers to a polymer of two or more amino acids.

The term “amino acid” as used herein refers to a compound that contains in its monomeric state at least an amine (—NH₂) and a carboxyl (—COOH) functional group. Two amino acids can be covalently bonded to one another by a peptide bond. If an amino acid is conjugated to another amino acid through a pseudopeptide bond, as described below, the amine or carboxyl group may be replaced by other chemical moieties depending on the nature of the pseudopeptide bond. Preferably, the term “amino acid” as used herein refers to alpha- or beta-amino acids. As used herein the term “amino acid” includes the proteinogenic amino acids alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); valine (Val) and selenocysteine (Sec). The term “amino acid” as used herein also includes unnatural amino acids.

“Unnatural amino acids” in the sense of the present application are non-proteinogenic amino acids that occur naturally or are chemically synthesized, for example, norleucine (Nle), methoxinine, homopropargylglycin, ornithine, norvaline, homoserine, and other amino acid analogues such as those described in Liu C C, Schultz P G, Annu Rev Biochem 2010, 79: 413-444 and Liu D R, Schultz P G, Proc Natl Acad Sci U S A 1999, 96: 4780-4785. An unnatural amino acid, as used herein, may be, for instance, a proteinogenic amino acid in D-form, for instance, DGIu. Additional contemplated unnatural amino acids are para-, ortho- or meta-substituted phenylalanine, such as para-ethynylphenylalanine, 4-Cl-phenylalanine (Cpa), 4-amino-phenylalanine, and 4-NO₂-phenylalanine, homoprolin, homoalanine, beta-alanine, 1-naphthylalanine (1Nal), 2-naphtylalanine (2Nal), p-benzoyl-phenylalanine (Bpa), biphenylalanine (Bip), homophenylalanine (hPhe), homopropargylglycine (Hpg), azidohomoalanine (Aha), cyclohexylalanine (Cha), aminohexanoic acid (Ahx), 2-aminobutanoic acid (Abu), azidonorleucine (Anl), tert-leucine (Tle), 4-amino-carbamoyl-phenylalanine (Aph(Cbm)), 4-amino-hydroorotyl-phenylalanine (Aph(Hor)), S-Acetamidomethyl-L-cysteine (Cys(Acm)), 3-benzothienylalanine, 4-amino-3-hydroxy-6-methylheptanoic acid (Sta). Some of these unnatural amino acids have been explored already for somatostatin and bombesin analogues (Fani M et al., J Nucl Med 2011, 52: 1110-1118; Ginj M et al., Proc Natl Acad Sci U S A 2006, 103: 16436-16441; Ginj M et al., Clin Cancer Res 2005, 11: 1136-1145; Mansi R, J Med Chem 2015, 58: 682-691).

As used herein, the term “hydrophobic amino acid” refers to amino acids that have a net zero charge at physiological pH (about pH 7.4). Hydrophobic amino acids can be proteinogenic hydrophobic amino acids or unnatural hydrophobic amino acids. Proteinogenic hydrophobic amino acids are for example serine, threonine, cysteine, selenocysteine, glycine, proline, alanine, valine, isoleucine, leucine, methionine, phenylalanine, tyrosine or tryptophan. Preferred proteinogenic hydrophobic amino acids are proline, isoleucine, leucine, phenylalanine, tyrosine and tryptophan. Unnatural hydrophobic amino acids are, for example, norleucine (Nle), methoxinine, tert-leucine (Tle), 1-naphthylalanine (1Nal), 2-naphtylalanine (2Nal), 3-benzothienylalanine, p-benzoyl-phenylalanine (Bpa), biphenylalanine (Bip), homophenylalanine (hPhe), homopropargylglycine (Hpg), azidohomoalanine (Aha), cyclohexylalanine (Cha), aminohexanoic acid (Ahx), 2-aminobutanoic acid (Abu), azidonorleucine (Anl), 2-aminooctynoic acid (Aoa), norvaline (Nva), para-ethynylphenylalanine, 4-Cl-phenylalanine, homoproline and homoalanine.

In some embodiments the peptidomimetic of the present invention has a cellular uptake (i.e. binding to the cell membrane and internalization into the cells) to a degree of at least about 10%, about 20%, about 30%, about 40%, about 50%, about 60%, about 70%, about 80%, about 90%, or about 95% of the total activity in an assay as described in Example 2.

In some embodiments, the peptidomimetic of the present invention specifically binds to CCK2R. The binding affinity of the peptidomimetic of the invention can be at least about 2%, about 10%, about 20%, about 30%, about 40%, about 50%, about 60%, about 70%, about 80%, about 90%, about 95%, or about 100% of the binding affinity of CCK8, minigastrin or pentagastrin for CCK2R. In some embodiments of the present invention the binding affinity of the peptidomimetic can even be higher than the binding affinity of CCK8, minigastrin or pentagastrin for CCK2R, for example, at least about 110%, about 120%, about 130%, about 140%, about 150%, about 160%, about 170%, about 180%, about 190%, about 200%, about 500%, about 1000%, about 1500%, or about 2000% of the binding affinity of CCK8, minigastrin or pentagastrin for CCK2R in an assay as described in Example 4 of WO 2018/224665 A1.

Specific binding to CCK2R in the context of the present invention means binding of the peptidomimetic of the present invention to CCK2R that can be displaced with a cognate ligand of CCK2R, such as gastrin, or a radiolabelled variant of a cognate ligand, such as [¹²⁵I]Tyr¹²-labelled gastrin-I. The half-maximal inhibitory concentration (IC50) is a measure of this specific binding as explained above and can be obtained from an assay as described in Example 4 of WO 2018/224665 A1.

Binding affinity according to the present invention is determined by measuring the half maximal inhibitory concentration (1050), wherein binding affinity and IC50 value have an inverse relationship, meaning that binding affinity increases with decreasing IC50 value and binding affinity decreases with increasing 1050 value. Therefore, a binding affinity of about 50% means that the IC50 value is about twice as high as the IC50 value of CCK8, minigastrin or pentagastrin for CCK2R.

The term “charged amino acid” as used herein includes amino acids that have non-zero net charge at a physiological pH of about 7.4 and includes proteinogenic and unnatural amino acids, for instance, the proteinogenic amino acids Arg, Lys, His, Glu, and Asp.

The structure of the peptidomimetic is indicated in the three-letter amino acid code known to the person skilled in the art, starting with the N-terminus (amino terminus) of the amino acid sequence βAla-Trp-(NMe)Nle-Asp-1Nal of the peptidomimetic on the left and ending with the C-terminus of the peptidomimetic on the right. For example, if the Linker in the structure X-Linker-βAla-Trp-(NMe)Nle-Asp-1Nal, e.g. DOTA-Linker-βAla-Trp-(NMe)Nle-Asp-1Nal, does not comprise an amino acid, the amino acid βAla forms the N-terminus of the peptidomimetic. In the structure X-Linker-βAla-Trp-(NMe)Nle-Asp-1Nal, 1Nal forms the C-terminus, which may be preferably amidated.

The chemical bond that connects two amino acids of the peptidomimetic of the present invention, such as of the amino acids in the sequence βAla-Trp-(NMe)Nle-Asp-1Nal, may be a peptide bond, i.e., an amide bond (—CONH—). A peptide bond as used herein can be formed between an amino group attached to the alpha carbon of one amino acid and the carboxyl group attached to the alpha carbon of another amino acid. A peptide bond may also form between an amino group and a carboxyl group, one of which is not attached to the alpha carbon of the amino acid, but to the side chain of the amino acid (isopeptide bond), for example the amino group in the side chain of lysine. The chemical bond that connects two amino acids may also be a pseudopeptide bond. In preferred embodiments, the chemical bond that connects two amino acids of the peptidomimetic (“—”) is an amide bond, unless otherwise indicated.

The term “pseudopeptide bond” as used herein refers to a bond that connects two amino acids and is not an amide bond (—CONH—). A pseudopeptide bond may also be included in the amidated C-terminus. Any pseudopeptide bond known in the art is contemplated in the context of the present invention, for instance, —CH₂NH—, —CONRCH₂—, —CONCH₃— (the latter also referred to as N-Me), or —CONR—, wherein R is alkyl, preferably methyl, ethyl, n-propyl, isopropyl, n-butyl, s-butyl, isobutyl, t-butyl, n-pentyl, 1-methylbutyl, 2-methylbutyl, 3-methylbutyl, 1,1-dimethylpropyl, 1-methyl-2-methylpropyl, 2,2-dimethylpropyl, or 1-ethylpropyl.

Herein, the identity of the chemical bond between two amino acids may be indicated in parentheses or square brackets in between the amino acids that are connected through the bond, for instance, Trp-(NMe)Nle. In this case, the two amino acids Trp and Nle are connected by a pseudopeptide bond of the structure —CONCH₃—, wherein the carboxyl group of Trp reacted with the amino group of Nle in a condensation reaction under release of one molecule water and the resulting amide nitrogen is methylated. The following spellings, exemplarily given for two amino acids Trp and Nle and the pseudopeptide bond N-Me, are interchangeably used herein to indicate the nature of the pseudopeptide bond: “Trp-(N-Me)-Nle”, “Trp-(N-Me)Nle” and “Trp(N-Me)-Nle”. Alkylester, alkylether and urea bonds are also contemplated as pseudopeptide bonds. Other pseudopeptide bonds such as 1,2,3-triazoles (Mascarin A et al., Bioconjug Chem 2015, 26: 2143-2152) or other amide bond bioisosters, which have shown to stabilize peptidomimetics and improve tumour targeting are also contemplated.

In some embodiments the presence of a pseudopeptide bond is indicated by the term “psi”, as commonly used in the art. For instance, X⁴-psi[CH₂NH]-X⁵ indicates that the two amino acids X⁴ and X⁵ are connected via the pseudopeptide bond —CH₂NH—. In some embodiments the pseudopeptide bond can be -psi[CH₂—NH—CO—NH]—, -psi[CH₂—NH]—, -psi[CH₂—CH₂]—, -psi[CS—NH]— or -psi[Tz]-.

In some embodiments the pseudopeptide bonds are: —COO—, —COS—, —COCH₂—, —CSNH—, —CH₂CH₂—, —CHCH—, —CC—, —NHCO—, —CH₂S—, —CH₂—NH—CO—NH— and —CH₂O—.

In the context of the present invention L- and D-amino acids are equally contemplated. Any amino acid of the present invention may be present in L- or D-form unless otherwise stated. The L-from is indicated by reciting a “L” directly before the name of the amino acid, the D-form is indicated by reciting a “D” directly before the name of the amino acid. For instance, “DGIu” refers to the D-form of the amino acid glutamate and “LGlu” refers to the L-form of the amino acid glutamate. In preferred embodiments, the enantiomeric form of one, more or all of the amino acids of the peptidomimetic is the L-form. For instance, if the nomenclature encompasses both enantiomeric forms, for example, as is the case in “Asp”, then the preferred enantiomeric form is the L-form (as in “LAsp”).

The term “chelator” is used as in the art and refers to organic compounds that are polydentate ligands that form two or more coordinate bonds with a metal atom.

In preferred embodiments of the present invention the C-terminus (carboxy terminus) of the peptidomimetic is modified to, for example, reduce proteolytic degradation, increase shelf life, and/or improve cellular uptake. The C-terminus may be for example amidated with an —NR′R″ group, wherein R′ and R″ are independently hydrogen or a substituted or non-substituted alkyl as defined herein. In some embodiments R′ and R″ are independently ethyl, propyl, butyl, pentyl or hexyl. In a preferred embodiment the peptidomimetic of the present invention is amidated at the C-terminus with a —NH₂ group. The C-terminus can also be modified with an ester of the type —C(O)OR′″, wherein R′″ can be a substituted or non-substituted alkyl as defined herein, for instance, ethyl, propyl, butyl, pentyl or hexyl.

The term “alkyl” as used herein refers to a straight-chain or branched saturated aliphatic hydrocarbon with 1 to 20 (C1-C20), preferably 1 to 15 (C1-C15), more preferably 1 to 10 (C1-C10), and most preferably 1 to 5 (C1-C5) carbon atoms. For example, “alkyl” may refer to methyl, ethyl, n-propyl, isopropyl, n-butyl, s-butyl, isobutyl, t-butyl, n-pentyl, 1-methylbutyl, 2-methylbutyl, 3-methylbutyl, 1,1-dimethylpropyl, 1-methyl-2-methylpropyl, 2,2-dimethylpropyl, and 1-ethylpropyl.

The “alkyl” group may be substituted with, for example, halogens, such as fluorine, chlorine and bromine, amines, such as a primary amine (—NH₂), primary amide, hydroxyl (—OH), further oxygen-, sulfur- or nitrogen-containing functional groups, heterocycles, or aryl substituents, such as phenyl and naphthyl.

The peptidomimetic of the present invention comprises 5 to 10 amino acids. The term “comprises 5 to 10 amino acids” as used herein means that the peptidomimetic does not have more than 10 amino acids and not less than 5 amino acids.

Particularly preferred are peptidomimetics that comprise 5, 6, 7, 8, 9 or 10 amino acids. More preferred are peptidomimetics that comprise 7 amino acids.

In some embodiments of the present invention the peptidomimetic comprises the chelator DOTA that coordinates a radionuclide, for example a metal radionuclide, such as 225 AC, ²¹²Bi, ²¹³Bi, ⁶²Cu, ⁶⁴Cu, ⁶⁷Cu, ⁶⁹Cu, ⁶⁶Ga, ⁶⁷Ga, ⁶⁸Ga, ¹¹¹In, ^{113m}In, ¹⁷⁷Lu, ¹⁸⁶Re, ¹⁸⁸Re, ⁴³Sc, ⁴⁴Sc, ⁴⁷Sc, ¹⁵⁵Tb, ¹⁶¹Tb, ^{99m}Tc, ⁸⁶Y, ⁹⁰Y, ¹⁶⁹Yb, or ¹⁷⁵Yb.

In particularly preferred embodiments of the present invention, the DOTA group of the peptidomimetic coordinates the radionuclide ⁹⁰Y, ¹¹¹In, ⁶⁸Ga or ¹⁷⁷Lu.

In some embodiments of the present invention, the radionuclide has no therapeutic effect, such as a cytotoxic effect. In some embodiments, the radionuclide does at least not have a cytotoxic effect to a degree that is therapeutically relevant. The skilled person is able to determine an administered dose/radioactivity dose of the radionuclide that is sufficient for being detected, for instance in a method of imaging, but low enough for not having a therapeutic effect. Consequently, in some embodiments of the present invention, for instance, the method of imaging and any diagnostic uses or methods, a dose of the radionuclide is used that is sufficient for detection, but does not have a therapeutic effect. As used herein, a radionuclide without therapeutic effect is referred to as “non-therapeutic radionuclide”. Thus, the present invention also relates to non-therapeutic embodiments of the radionuclide. For example, the invention includes non-therapeutic radionuclides.

Preferred non-therapeutic radionuclides, which can be used for imaging, are ⁶⁴Cu, ⁶⁷Ga, ⁶⁸Ga, ¹²³I, ¹²⁴I, ¹²⁵I, ¹³¹I, ¹¹¹In, ¹⁷⁷Lu, ²⁰³Pb, ⁹⁷Ru, ⁴⁴Sc, ¹⁵²Tb, ¹⁵⁵Tb, ^{99m}Tc, ¹⁶⁷Tm, ⁸⁶Y, and ⁸⁹Zr.

### Cytotoxic group

In some embodiments, the peptidomimetic comprises a cytotoxic group. The term “cytotoxic group” as used herein refers to any material or chemical moiety that directly or indirectly causes the death of the cell to which the peptidomimetic that comprises the cytotoxic group is bound or by which it has been internalized.

The cytotoxic group can be, for example, a chemotherapeutic agent or a radionuclide. If the chemotherapeutic agent or radionuclide that is comprised by the peptidomimetic is internalized by a cell that expresses CCK2R, the CCK2R expressing cell is killed by the chemotherapeutic agent or radionuclide. In some embodiments, the cell to which the peptidomimetic is bound may also be killed without internalizing the peptidomimetic that comprises the cytotoxic group.

The chemotherapeutic agent can be selected from the group consisting of vinblastine monohydrazide, tubulysin B hydrazide, actinomycin, all-trans retinoic acid, azacitidine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, indotecan, indimitecan, mertansine, emtansine, valrubicin, vemurafenib, vinblastine, vincristine, vindesine, and vinorelbine.

Radionuclides that can be used include metal and halogen radionuclides. The radionuclide of the present invention can be for example selected from radioisotopes of P, Sc, Cr, Mn, Fe, Co, Cu, Zn, Ga, As, Br, Sr, Y, Tc, Ru, Rh, Pd, Ag, In, Sn, Sb, Te, I, Pr, Pm, Sm, Gd, Tb, Y, Ho, Er, Lu, Ta, W, Re, Os, Ir, Au, Hg, Tl, Pb, Bi, Po, At, Ra, Ac, Th, and Fm. Preferred radionuclides include, but are not limited to, ²²⁵Ac, ¹¹¹Ag, ⁷⁷As, ²¹¹At, ¹⁹⁸Au, ¹⁹⁹Au, ²¹²Bi, ²¹³Bi, ⁷⁷Br, ⁵⁸Co, ⁵¹Cr, ⁶⁷Cu, ¹⁵²Dy, ¹⁵⁹Dy, ¹⁶⁵Dy, ¹⁶⁹Er, ²⁵⁵Fm, ⁶⁷Ga, ¹⁵⁹Gd, ¹⁹⁵Hg, ¹⁶¹Ho, ¹⁶⁶Ho, ¹²³I, ¹²⁵I, ¹³¹I, ¹¹¹In, ¹⁹²Ir, ¹⁹⁴Ir, ¹⁹⁶Ir, ¹⁷⁷Lu, ^{189m}Os, ³²P, ²¹²Pb, ¹⁰⁹Pd, ¹⁴⁹Pm, ¹⁴²Pr, ¹⁴³Pr, ²²³Ra, ¹⁸⁶Re, ¹⁸⁸Re, ¹⁰⁵Rh, ¹¹⁹Sb, ⁴⁷Sc, ¹⁵³Sm, ^{117m}Sn, ¹²¹Sn, ⁸⁹Sr, ¹⁴⁹Tb, ¹⁶¹Tb, ^{99m}Tc, ¹²⁷Te, ²²⁷Th, ²⁰¹Tl, and ⁹⁰Y.

### Photosensitizer

In some embodiments of the present invention the peptidomimetic comprises a photosensitizer.

As used herein the term “photosensitizer” refers to a material or chemical moiety that becomes toxic or releases toxic substances upon exposure to light such as singlet oxygen or other oxidizing radicals which are damaging to cellular material or biomolecules, including the membranes of cells and cell structures, and such cellular or membrane damage may eventually kill the cells. Photosensitizers as defined herein are known in the art and available to the skilled person. The cytotoxic effects of photosensitizers can be used in the treatment of various abnormalities or disorders, including neoplastic diseases. Such treatment is known as photodynamic therapy (PDT) and involves the administration of a photosensitizer to the affected area of the body, followed by exposure to activating light in order to activate the photosensitizer and convert them into cytotoxic form, whereby the affected cells are killed or their proliferative potential diminished.

Photosensitizers exert their effects by a variety of mechanisms, directly or indirectly. Thus for example, certain photosensitizers become directly toxic when activated by light, whereas other photosensitizers act to generate toxic species, e.g. oxidizing agents such as singlet oxygen or oxygen-derived free radicals, which are destructive to cellular material and biomolecules, such as lipids, proteins and nucleic acids, and ultimately kill cells.

In some embodiments the photosensitizer includes, for example, psoralens, porphyrins, chlorins and phthalocyanines. Porphyrin photosensitizers act indirectly by generation of toxic oxygen species and are particularly preferred. Porphyrins are naturally occurring precursors in the synthesis of heme. In particular, heme is produced when iron (Fe²⁺) is incorporated in protoporphyrin IX (PpIX) by the action of the enzyme ferrochelatase. PpIX is a highly potent photosensitizer. Further photosensitizer that can be used in the context of the present invention are aminolevulinic acid (ALA), silicon phthalocyanine Pc 4, m-tetrahydroxyphenylchlorin (mTHPC) and mono-L-aspartyl chlorin e6 (NPe6), porfimer sodium, verteporfin, temoporfin, methyl aminolevulinate, hexyl aminolevulinate, laserphyrin-PDT, BF-200 ALA, amphinex and azadipyrromethenes.

### Linker

The peptidomimetic of the present invention comprises an amino acid polymer with the sequence βAla-Trp-(NMe)Nle-Asp-1Nal. The total number of amino acids comprised by the peptidomimetic is limited as defined herein. In addition to the amino acid polymer βAla-Trp-(NMe)Nle-Asp-1Nal, the peptidomimetic of the present invention comprises a Linker and group X as defined herein. X is a chelator comprising a radionuclide or a prosthetic group comprising a radionuclide.

The Linker connects group X and the amino acid polymer with the sequence βAla-Trp-(NMe)Nle-Asp-1Nal. Preferably the Linker forms a covalent bond with the sequence 8Ala-Trp-(NMe)Nle-Asp-1Nal as well as with group X. In some embodiments the Linker forms an amid bond with the chelator, the prosthetic group or the sequence βAla-Trp-(NMe)Nle-Asp-1Nal. In some embodiments the Linker forms an amide bond with the chelator and the sequence βAla-Trp-(NMe)Nle-Asp-1Nal. In some embodiments the Linker forms an ester bond with group X.

In some embodiments the Linker forms an amide bond with the amino group of beta-Ala of the amino acid polymer of the sequence βAla-Trp-(NMe)Nle-Asp-1Nal.

In some embodiments the Linker is a bifunctional molecule which can form a covalent bond with the amino group of the beta-Ala of the sequence βAla-Trp-(NMe)Nle-Asp-1Nal on the one side and group X, e.g. DOTA, on the other side under conditions that are amendable to solid phase peptide synthesis.

In some embodiments the Linker is a small organic molecule with a molecular weight below 1000 Da, below 900 Da, below 800 Da, below 700 Da, below 600 Da, below 500 Da, below 400 Da, below 300 Da, below 200 Da or below 100 Da. In some embodiments, the molecular weight of the Linker is between 50 and 300 Da, between 50 and 400Da, between 50 and 500 Da, between 50 and 600 Da, between 50 and 700 Da, between 50 and 800 Da, between 50 and 900 Da or between 50 and 1000 Da.

Preferably the Linker does not perturb the specific binding of the amino acid polymer with the sequence βAla-Trp-(NMe)Nle-Asp-1Nal to CCK2R.

The Linker can serve as a pharmacokinetic modifier influencing, for example, the hydrophilicity and pharmacokinetics of the peptidomimetic. For instance in some embodiments the Linker may increase the kidney-to-tumour ration of the peptidomimetic.

The Linker may be a natural or unnatural amino acid, such as Gly, Ala, Gln, Glu, His, all in L- or D-form, or an amino acid polymer consisting of one or more of these amino acids, or any other chemical moiety, such as polyethylene glycol or a carbohydrate, as well as aminoalkanonyl, for instance, aminohexanoyl or aminobenzoyl or piperidine moieties. In some embodiments the Linker can be 6-aminohexanoic acid, 2-aminobutanoic acid, 4-aminobutyric acid, 4-amino-1-carboxymethylpiperidine or urea or another chemical moiety that allows introducing a functional group in the peptidomimetic. Preferred molecules that may be comprised in the Linker are y-amino-butyric acid (GABA), γ-amino-β-hydroxybutyric acid (GABOB) or DGlu.

In some embodiments the Linker is a combination of the above mentioned Linkers. For instance, the Linker may comprise or consist of two, three, four or five of the above-mentioned molecules, such as amino acids. For instance, the Linker may consist of two molecules 6-aminohexanoic acid, 2-aminobutanoic acid, 4-aminobutyric acid or DGIu. In some embodiments the Linker is further functionalized with hydroxyl-groups or carboxyl-groups to increase the hydrophilicity of the Linker. For instance, the aminoalkanonyl group may be substituted with one or more hydroxyl or carboxyl-groups.

In some embodiments the Linker is GABOB-GABOB, which refers to two molecules of γ-amino-β-hydroxybutyric acid, which are condensed through an amide bond. In some embodiments the Linker is GABA-GABA, which refers to two molecules of γ-amino-butyric acid, which are condensed through an amide bond. In some embodiments the Linker is gamma-DGlu-gamma-DGlu (γDGlu-γDGlu).

As used herein, “pharmacokinetic modifier” means a chemical moiety that influences the pharmacokinetics of the peptidomimetic, such as the hydrophilicity, biodegradation, and clearance. For instance, the pharmacokinetic modifier may increase the half-life of the peptidomimetic in the blood stream.

### Chelator and Prosthetic Group

For the introduction of the radionuclide, the peptidomimetic of the invention comprises a chelator or a prosthetic group. The chelator or prosthetic group is bound to the radionuclide and can be targeted to cells, e.g. cancer cells, such as tumour cells, via the peptidomimetic's affinity for the CCK2R receptor. The chelator or prosthetic group is linked to the sequence βAla-Trp-(NMe)Nle-Asp-1Nal via the Linker.

The introduction of the radionuclide into the chelator or prosthetic group can be performed before or after conjugation of the chelator or prosthetic group with the amino acid polymer of the sequence βAla-Trp-(NMe)Nle-Asp-1Nal via the Linker.

In some embodiments the chelator coordinates a metal radionuclide as mentioned herein.

The chelator may contain different donor groups for metal complexation such as oxygen, nitrogen, sulphur, (carboxyl, phosphonate, hydroxamate, amine, thiol, thiocarboxylate or derivatives thereof) and comprises acyclic and macrocyclic chelators such as polyaminopolycarboxylic ligands.

In some embodiments the chelator is selected from the group consisting of diethylenetriaminopentaacetic acid (DTPA), ethylenediaminetetraacetic acid (EDTA), 1,4,7-triazacyclononane-1,4,7-tris[methylene(2-carboxyethyl)]phosphinic acid (TRAP), 1,4,7,10-tetraazacyclododecane-1,4,7,10-tetraacetic acid (DOTA), 1,4,7-triazacyclononane-1,4,7-triacetic acid (NOTA), 1,4,7-triazacyclononane-1,4-diiacetic acid (NODA), 1,4,8,11-tetraazacyclotetradecane-1,4,8,11-tetraacetic acid (TETA) as well as derivatives thereof such as DOTA or NOTA functionalized with a glutaric acid arm (DOTAGA, NOTAGA). Other chelators are also contemplated in particular chelators for chelating radiometals.

Further chelators that are contemplated, for example for chelating ^{99m}Tc, include, but are not limited to, diamidedithiols (N₂S₂), triamidethiols (N₃S), tetraamines (N₄) and hydrazinonicotinic acid (HYNIC). HYNIC is usually used in combination with co-ligands to complete the coordination sphere of the metal, which include and are not limited to ethylendiamine-N,N′-diacetic acid (EDDA) and tricine. In some embodiments, using the organometallic aqua ion ^{99m}Tc(CO)₃(H₂O)₃, tricarbonyl complexes can be generated by exchanging water molecules with mono-, di- and tridentate chelators to form stable complexes including also the click-to-chelate methodology.

Further chelators, which are for example useful for labelling the peptidomimetics with ⁶⁸Ga, include, but are not limited to N,N′-bis[2-hydroxy-5-(carboxyethyl)benzyl]ethylenediamine-N,N′-diacetic acid (HBED-CC), siderophore-based ligands such as desferrioxamine, hydroxypyridinone ligands such as deferiprone and tris(hydroxypyridinone) (THP), and derivatives thereof.

In some embodiments the chelator is

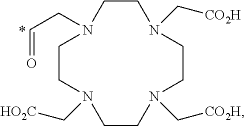 [SMILES: O=CCN1CCN(CC(=O)O)CCN(CC(=O)O)CCN(CC(=O)O)CC1]

wherein the asterisk indicates the position where the chelator is directly bound to the Linker. In some embodiments

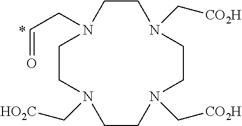 [SMILES: O=CCN1CCN(CC(=O)O)CCN(CC(=O)O)CCN(CC(=O)O)CC1]

is bound to a nitrogen or oxygen atom of the Linker. For instance, the carbonyl carbon marked with the asterisk may be bound to an amine of the Linker thereby forming an amide bond between the chelator and the Linker. In some embodiments

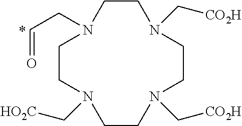 [SMILES: O=CCN1CCN(CC(=O)O)CCN(CC(=O)O)CCN(CC(=O)O)CC1]

is bound to the amine of GABA, GABOB or DGIu of the Linker.

A preferred chelator is DOTA or derivatives thereof, such as, DO2A, DO3AM^{nBu}, DO3AM^{en}, DO3AM^{pNO2Bn}, Do3AM^{C5H12-CO2H}, DOTAM, DTMA and DOTA-(gly)₄.

Preferred prosthetic groups of the present invention are labelled with radionuclides of halogens such as iodine, bromine or fluorine, for example those mentioned herein. In some embodiments the prosthetic group will be selected from a group consisting of the Bolton-Hunter reagent, N-succinimidyl-5-(trialkylstannyl)-3-pyridinecarboxylates or N-succinimidyl-4-[¹³¹I]iodobenzoate ([¹³¹I]SIB) for radioiodination. In some embodiments the prosthetic group will be selected from a group comprising but not limited to 4-[¹⁸F]fluorophenacyl bromide, N-succinimidyl-4-[¹⁸F]fluorobenzoate ([¹⁸F]SFB), N-succinimidyl-4-([¹⁸F]fluoromethyl)benzoate, 4-[¹⁸F]fluorobenzaldehyde, 6-[¹⁸F]fluoronicotinic acid tetrafluorophenyl ester ([¹⁸F]F-Py-TFP), silicon-containing building blocks such as N-Succinimidyl 3-(di-tert-butyl[¹⁸F]fluorosilyl)benzoate ([¹⁸F]SiFB), carbohydrate-based prosthetic groups, such as [¹⁸F]fluoro-deoxyglucose, preferably 2-[¹⁸F]fluoro-2-deoxyglucose ([¹⁸F]FDG), and [¹⁸F]fluoro-deoxymannose, preferably [¹⁸F]-fluoro-2-deoxymannose, or derivatives thereof, maleimide-based and heterocyclic methylsulfone-based ¹⁸F-synthons, ¹⁸F-labelled prosthetic groups such as ¹⁸F-azides or ¹⁸F-alkynes permitting labelling via click chemistry, ¹⁸F-labelled organotrifluoroborates and [¹⁸F]fluoropyridines. In some embodiments a chelator-based labelling approach using aluminum-fluoride (Al¹⁸ F) is applied for radiofluorination.

In some embodiments, the radiohalide that is bound to the prosthetic group is selected from the group consisting of ¹²³I, ¹²⁴I, ¹²⁵I, ¹³¹I, and ¹⁸F.

### Further Aspects and Embodiments of the Invention

The present invention also relates to a method of producing the present peptidomimetics as described herein. Production of the present peptidomimetics is possible by standard organic chemistry methods and solid phase peptide synthesis methods available to the person skilled in the art. The method at least comprises synthesizing the amino acid polymer of the peptidomimetic (Behrendt R et al., J Pept Sci 2016, 22: 4-27; Jones J, Amino Acid and Peptide Synthesis, Oxford University Press, New York 2002; Goodman M, Toniolo C, Moroder L, Felix A, Houben-Weyl Methods of Organic Chemistry, Synthesis of Peptides and Peptidomimetics, workbench edition set, Thieme Medical Publishers, 2004).

The present invention further relates to pharmaceutical compositions, for therapeutic of diagnostic use, comprising the peptidomimetic described herein and a pharmaceutically acceptable carrier. Pharmaceutical compositions according to the present invention can be used in the treatment of CCK2R related diseases, such as diseases that are characterized by CCK2R expression or overexpression. In some embodiments the pharmaceutical compositions of the present invention can be used in the treatment of cancer, in particular such cancers that are characterized by expression of CCK2R. In some embodiments of the present invention the pharmaceutical compositions of the present invention can be used to deliver a cytotoxic group, such as a chemotherapeutic agent or a radionuclide to CCK2R expressing tumour cells. Thus, pharmaceutical compositions of the present invention can be used for targeted cancer therapy.

The present invention also relates to a kit comprising one or more components of the present invention, for instance, the pharmaceutical composition according to the present invention or the peptidomimetic according to the present invention. The kit may further comprise an information leaflet that provides explanations how to prepare or use the peptidomimetic, pharmaceutical composition or diagnostic composition of the present invention. In some embodiments, the kit comprises the pharmaceutical composition of the present invention ready for use. In a further embodiment, the kit comprises two or more compositions that are sufficient to prepare the pharmaceutical composition, ready for use. For instance, in some embodiments, the kit may comprise a first composition comprising a peptidomimetic that comprises a chelator, and a second composition comprising a reporter or cytotoxic group. To prepare the final diagnostic or therapeutic composition, the skilled person would follow the information leaflet provided in the kit and combine the first and second composition to generate a ready to use diagnostic or therapeutic composition.

The compositions of the present invention can be used for diagnostic purposes. Diagnostic compositions of the present invention can be administered to the patient as part of the diagnostic process, for example, to allow imaging of CCK2R expressing cells or tissues, for example CCK2R expressing tumour cells. The diagnostic composition of the present invention can be used in methods of imaging, such as methods of imaging according to the present invention, for example methods of imaging tumour cells.

The present invention also relates to a use of the peptidomimetic of the present invention described herein for delivering the radionuclide to cells. Preferably, the radionuclide is delivered to a cell that expresses CCK2R, for example a cancer cell expressing CCK2R. The use of the present peptidomimetic can be in vivo or in vitro. For example, the peptidomimetic of the present invention can be used to deliver a reporter group or a cytotoxic group to a human or animal, for example a mammal, such as mouse, rat, rabbit, hamster or other mammals. In some embodiments of the present invention the peptidomimetic can be used to deliver a reporter group or a cytotoxic group to cells ex vivo, for example, immortalized or primary cell lines that are cultured in cell culture.

The present invention also relates to a method of imaging cells as described herein. The method of imaging cells described herein makes use of the peptidomimetic described herein. In some embodiments of the present invention, the method of imaging cells makes use of a non-therapeutic peptidomimetic as described herein. The present method of imaging cells may involve or can be based on established methods of imaging, such as computer tomography (CT), magnetic resonance imaging (MRI), scintigraphy, SPECT, PET, or other similar techniques. Based on the individual method of imaging that is used, the skilled person will select the proper radionuclide. The present method of imaging cells can be carried out in vivo or in vitro. In some embodiments of the present invention contacting a cell with the peptidomimetic of the present invention involves administering the peptidomimetic described herein to a patient, for example a patient that suffers from cancer, for example a cancer that involves the expression of CCK2R. In some preferred embodiments the cell is a tumour cell. Thus, in some preferred embodiments a tumour cell is contacted with the peptidomimetic. In some preferred embodiments, the tumour cell expresses CCK2R.

In some embodiments, the present invention also relates to a method of treating a patient that suffers from a disease that involves the expression of CCK2R, for example a cancer that is characterized by the expression of CCK2R in the tumour cells. Such a method of treating a patient involves the administration of the peptidomimetic of the present invention to the patient.

In some embodiments, the present invention relates to the peptidomimetic described herein for use in therapy. In preferred embodiments of the present invention the peptidomimetic is for use in the treatment of cancer. Preferably, the cancer is a cancer that expresses CCK2R on the surface of tumour cells.

The peptidomimetic of the present invention is useful for the diagnostic workup and treatment of various types of cancer, for example: thyroid cancer such as medullary thyroid carcinomas (MTC), lung cancers such as small cell lung cancer (SCLC), gastrointestinal stromal tumours, tumours of the nervous system such as astrocytomas and meningiomas, stromal ovarian cancers, gastrointestinal cancers, neuroendocrine tumours, gastroenteropancreatic tumours, neuroblastomas, tumours of the reproductive system such as breast carcinomas, endometrial carcinomas, ovarian cancers and prostate carcinomas, insulinomas, vipomas, bronchial and ileal carcinoids, leiomyosarcomas, leiomyomas and granulosa cell tumours. In some preferred embodiments the above mentioned types of cancer express CCK2R.

In some embodiments, the peptidomimetic does not comprise GABOB-GABOB-βAla-Trp-(NMe)Nle-Asp-1Nal-NH₂. In some embodiments, the peptidomimetic does not comprise DOTA-GABOB-GABOB-βAla-Trp-(NMe)Nle-Asp-1Nal-NH₂. In some embodiments, the peptidomimetic does not comprise ¹⁷⁷Lu-GABOB-GABOB-βAla-Trp-(NMe)Nle-Asp-1Nal-NH₂. In some embodiments, the peptidomimetic has the structure X-Linker-βAla-Trp-(NMe)Nle-Asp-1Nal as defined herein, with the proviso that the peptidomimetic does not comprise DOTA-GABOB-GABOB-βAla-Trp-(NMe)Nle-Asp-1Nal-NH₂.

In some embodiments of the invention the peptidomimetic has one of the following structures:

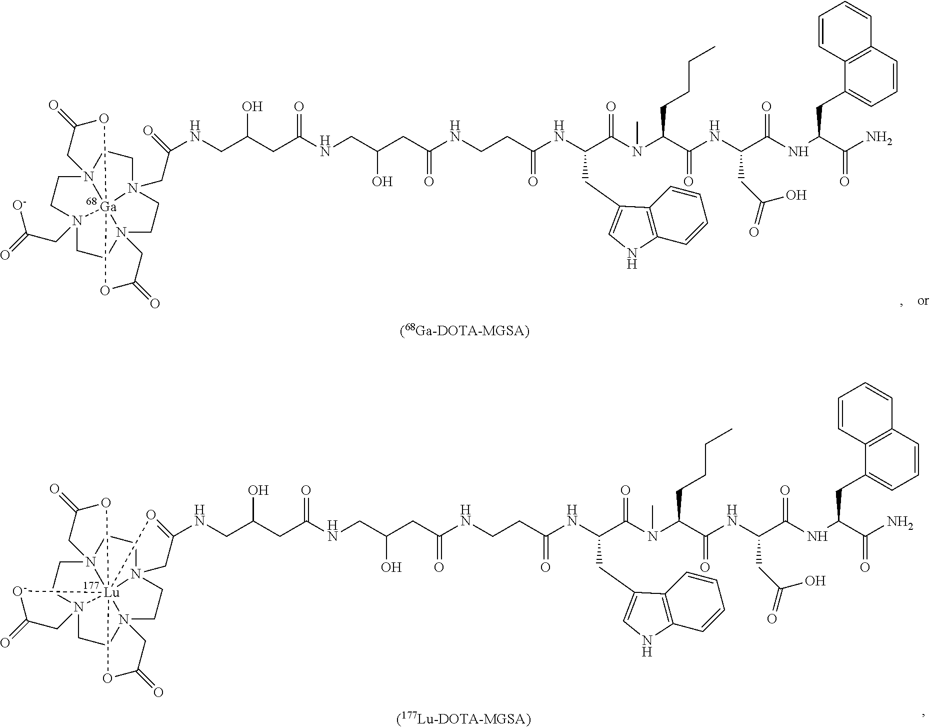

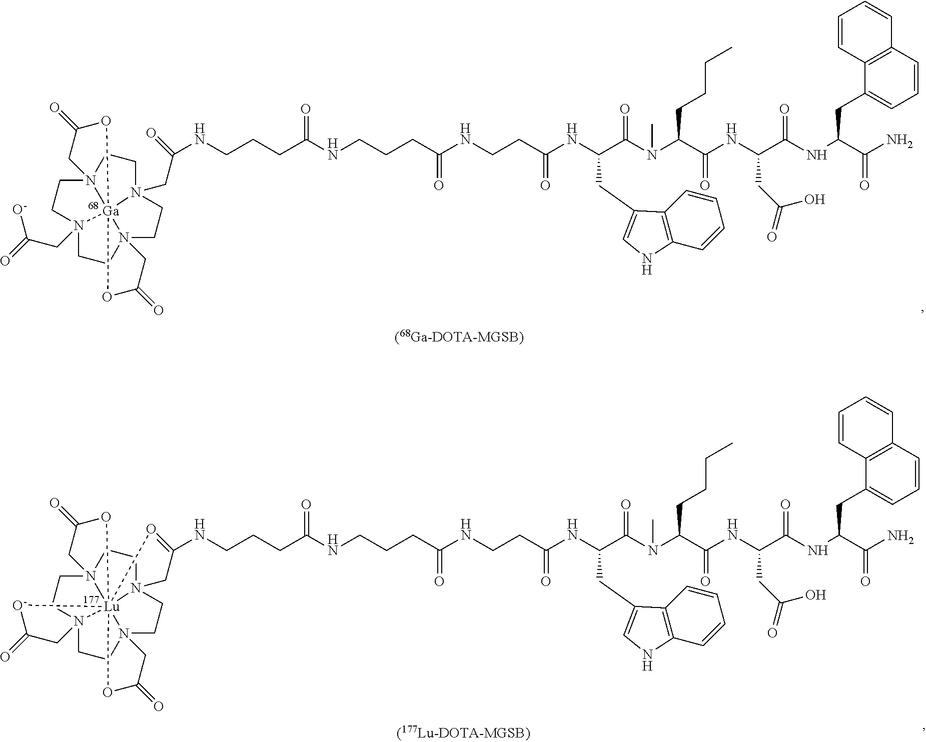

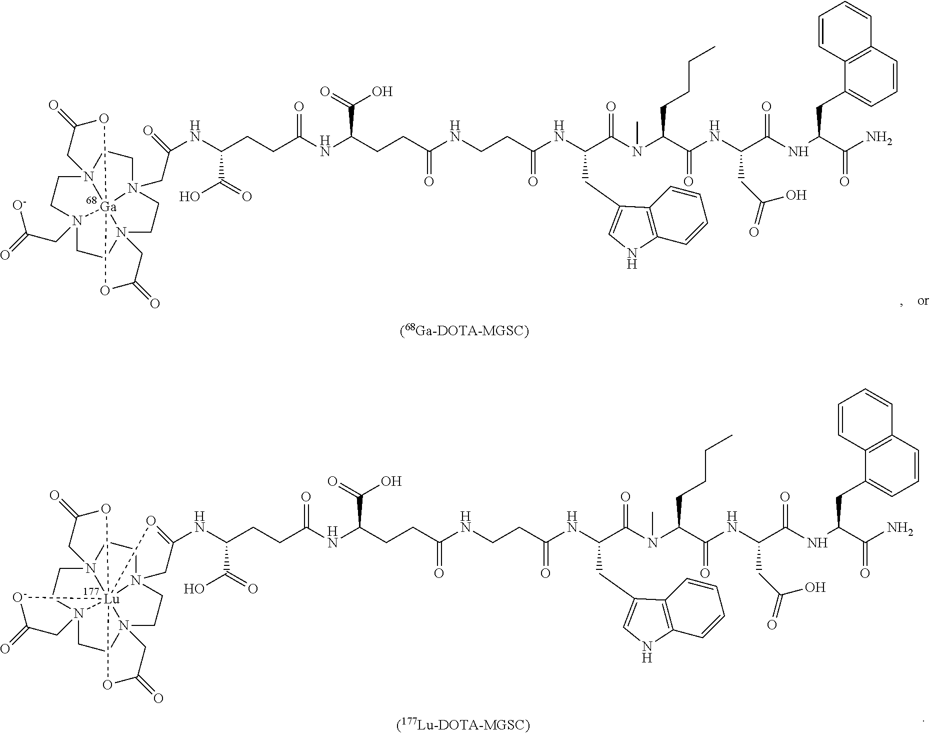

In some embodiments the peptidomimetic has the structure

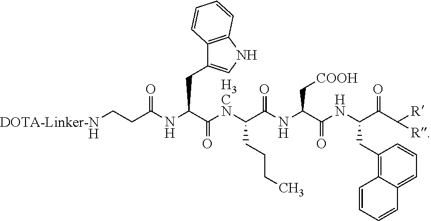

In some embodiments of the invention the Linker-βAla-Trp-(NMe)Nle-Asp-1Nal portion of the peptidomimetic is

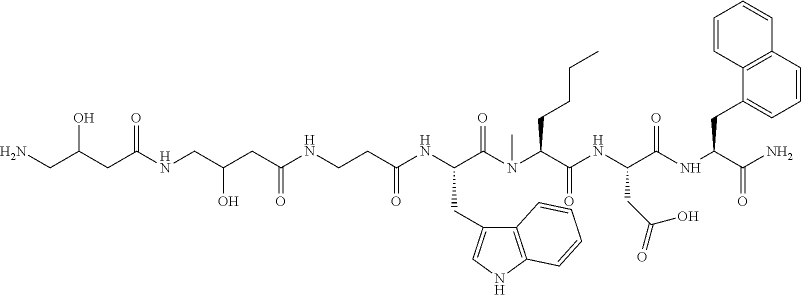 [SMILES: CCCC[C@@H](C(=O)N[C@@H](CC(=O)O)C(=O)N[C@@H](Cc1cccc2ccccc12)C(N)=O)N(C)C(=O)[C@H](Cc1c[nH]c2ccccc12)NC(=O)CCNC(=O)CC(O)CNC(=O)CC(O)CN]

that is bound to X via the N-terminal primary amine of the GABOB-GABOB Linker. In a preferred embodiment X is a chelator comprising a radionuclide.

In some embodiments of the invention the Linker-βAla-Trp-(NMe)Nle-Asp-1Nal portion of the peptidomimetic is

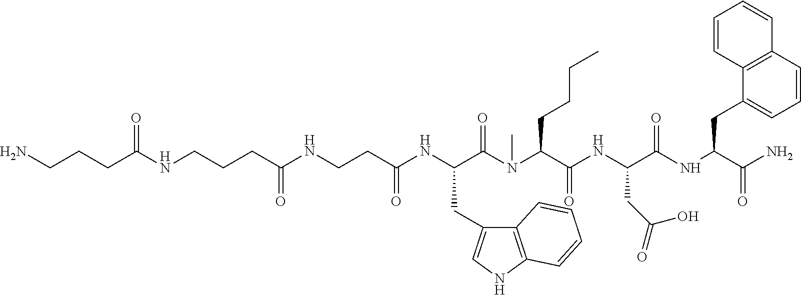 [SMILES: CCCC[C@@H](C(=O)N[C@@H](CC(=O)O)C(=O)N[C@@H](Cc1cccc2ccccc12)C(N)=O)N(C)C(=O)[C@H](Cc1c[nH]c2ccccc12)NC(=O)CCNC(=O)CCCNC(=O)CCCN]

that is bound to X via the N-terminal primary amine of the GABA-GABA Linker. In a preferred embodiment X is a chelator comprising a radionuclide.

In some embodiments of the invention the Linker-βAla-Trp-(NMe)Nle-Asp-1Nal portion of the peptidomimetic is

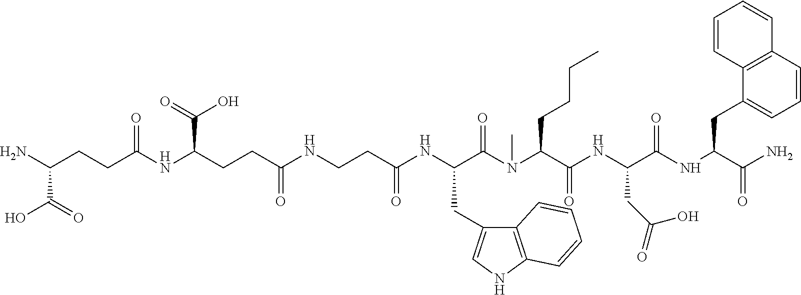 [SMILES: CCCC[C@@H](C(=O)N[C@@H](CC(=O)O)C(=O)N[C@@H](Cc1cccc2ccccc12)C(N)=O)N(C)C(=O)[C@H](Cc1c[nH]c2ccccc12)NC(=O)CCNC(=O)CC[C@@H](NC(=O)CC[C@@H](N)C(=O)O)C(=O)O]

that is bound to X via the N-terminal primary amine of the γDGIu-γDGIu Linker. In a preferred embodiment X is a chelator comprising a radionuclide.

### EXAMPLES

### Example 1: Synthesis of Peptidomimetics

The synthesis of the peptidomimetics of the present invention was performed using standard 9-fluorenylmethoxycarbonyl (Fmoc) chemistry.

The peptidomimetics were assembled on a Rink Amide MBHA resin (Novabiochem, Hohenbrunn, Germany) in N,N-Dimethylformamide (DMF) using an excess of Fmoc-protected amino acid, 1-Hydroxy-7-azabenzotriazole (HOAt), and (2-(7-Aza-1H-benzotriazole-1-yl)-1,1,3,3-tetramethyluronium hexafluorophosphate (HATU) in alkaline medium. The reactive side chains of the amino acids were masked with appropriate protection groups. After assembling the desired amino acid sequence, coupling of DOTA (tris(tBu) ester) was performed followed by cleavage of the peptidomimetic from the resin with concomitant removal of acid-labile protecting groups. After HPLC purification and lyophilization the peptidomimetics were obtained in ˜10% yield with a chemical purity ≥95% as confirmed by RP-HPLC and MALDI-TOF MS. Radiolabelling of the inventive peptidomimitics with different radiometals was performed using standard radiolabelling protocols by dissolving the peptide in aqueous solution such as 25-50% ethanol or PBS and mixing the solution with an acidic solution such as hydrochloric acid containing the radiometal and a solution such as sodium acetate solution or ascorbic acid solution for pH adjustment and incubating the mixture at high temperature (90-95° C.) for approximately 10-30 min. Radiolabelling with the different radiometals resulted in high labelling yields and radiochemical purity. HPLC analysis of the peptidomimetics and of the radiolabelled derivatives was performed on a Dionex chromatography system consisting of a Dionex UltiMate 3000 Pump (Dionex, Gemering, Deutschland), UV detection at 280 nm (UltiMate 3000 variable UV-detector) and radiodetection (Gabi Star, Raytest, Straubenhardt, Germany) and using a Phenomenex Jupiter 4 μ Proteo 90A 250×4.6 (C12) column and a flow rate of 1 mL/min together with a gradient system of water containing 0.1% TFA (solvent A) and acetonitrile containing 0.1% TFA (solvent B): 0-3 min 10% B, 3-18 min 10-55% B, 18-20 min 80% B, 20-21 min 10% B, 21-25 min 10%.

The following peptidomimetics (Table 1) were synthesized according to the above method:

TABLE 1

| Name | Structure |
|---|---|
| DOTA- | DOTA-Gabob-Gabob-βAla-Trp-(NMe)Nle-Asp-1Nal-NH₂ |
| MGSA | |
| DOTA- | DOTA-Gaba-Gaba-βAla-Trp-(NMe)Nle-Asp-1Nal-NH₂ |
| MGSB | |
| DOTA- | DOTA-γDGlu-γDGlu-βAla-Trp-(NMe)Nle-Asp-1Nal-NH₂ |
| MGSC | |
| DOTA- | DOTA-DGlu-Ala-Tyr-Gly-Trp-(NMe)Nle-Asp-1Nal-NH₂ |
| MGS5 | |

### Example 2: The Peptidomimetics Show High Cellular Internalization

These studies were carried out according to a previously published protocol (von Guggenberg E et al., Bioconjug Chem 2004, 15: 864-871) using 1.0 million of A431 cells transfected with the plasmid pCR3.1 containing the full coding sequence for the human CCK2R (A431-CCK2R) as well as the same cell line transfected with the empty vector alone (A431-mock) as a control, which were kindly provided by Dr. Luigi Aloj (Aloj L et al., J Nucl Med 2004, 45: 485-494). DMEM supplemented by 1% (v/v) fetal bovine serum was used as internalization medium and the non-specific cell uptake was studied in A431-mock cells instead of performing blocking studies. Additionally, cell internalization assays were performed using 1.5 million of rat pancreatic AR42J cells expressing rat CCK2R. RPMI supplemented by 1% (v/v) fetal bovine serum was used as internalization medium. Blocking experiments were carried out with 1 μM pentagastrin. The cells were incubated in triplicates with 10,000-500,000 cpm, for example 10,000-60,000 cpm of radiolabelled peptidomimetic (corresponding to a final concentration of 0.4 nM and ˜600 fmol of total peptidomimetic in the assay) and incubated at 37° C. for 2-4 h. The internalized fraction in A431-CCK2R and A431-mock cells as well as AR42J cells, was expressed in relation to the total activity added (% of total). For each radiolabelled peptide the mean value of one representative assay performed in triplicates is represented.

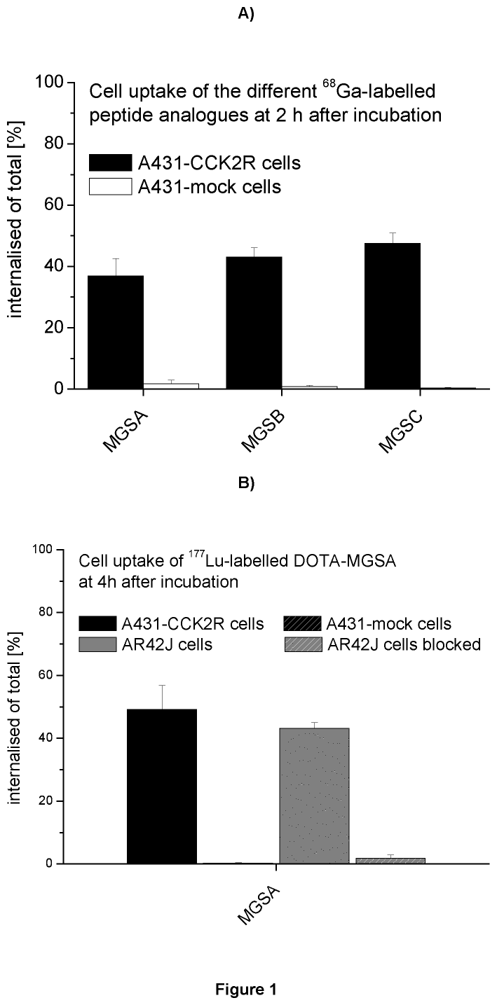

As shown in FIG. 1, a high receptor-specific cellular internalization was achieved by peptidomimetics conjugated to different linkers, such as GABOB-GABOB in MGSA, GABA-GABA in MGSB and γDGlu-γDGlu in MGSC. With the ⁶⁸Ga-labelled peptidomimetics after 2 h incubation on A431-CCK2R cells, a cellular internalization with values of 36.9±5.8% for ⁶⁸Ga-DOTA-MGSA, 43.1±3.1% for ⁶⁸Ga-DOTA-MGSB and 47.6±3.4% for ⁶⁸Ga-DOTA-MGSC, whereas the uptake in A431-mock cells was negligible (<2%). ¹⁷⁷Lu-DOTA-MGSA was studied in both cell lines with an uptake of 57.3±2.5 after 4h in A431-CCK2R cells, whereas <1% was observed in A431-CCK2R mock cells. Comparable uptake values were also found in AR42J cells with 45.2±0.8%. Receptor blocking with pentagastrin reduced the uptake to <1%.

### Example 3: The Peptidomimetics of the Present Invention have High Affinity to CCK2R

Affinity for CCK2R was studied in A431-CCK2R cells. The binding affinity was tested in a competition assay against [¹²⁵I][3-iodo-Tyr¹²,Leu¹⁵]gastrin-I in comparison with pentagastrin (Boc-β-Ala-Trp-Met-Asp-Phe-NH₂) for DOTA-MGSA, DOTA-MGSB and DOTA-MGSC. Radioiodination of gastrin-I was carried out using the chloramine-T method. Non-carrier-added [¹²⁵I][3-iodo-Tyr¹²,Leu¹⁵]gastrin-I was obtained by HPLC purification and stored in aliquots at −20° C. Binding assays were carried out in 96-well filter plates (MultiScreen_{HTS}-FB, Merck Group, Darmstadt, Germany) pretreated with 10 mM TRIS/139 mM NaCl pH 7.4 (2×250 μL). For the assay a number of 400,000 A431-CCK2R cells per well was prepared in 35 mM HEPES buffer pH 7.4 containing 10 mM MgCl₂, 14 μM bacitracin and 0.5% BSA (a hypotonic solution disturbing the integrity of the cell membranes). The cells were incubated in triplicates with increasing concentrations of the peptidomimetics (0.0003 to 1,000 nM) and [¹²⁵I-Tyr¹²]gastrin-I (˜25,000 cpm) for 1 h at RT. Incubation was interrupted by filtration of the medium and rapid rinsing with ice-cold 10 mM TRIS/139 mM NaCl pH 7.4 (2×200 μl) and the filters were counted in the gamma-counter. Half maximal inhibitory concentration (IC₅₀) values were calculated following nonlinear regression with Origin software (Microcal Origin 6.1, Northampton, MA) and a representative assay was chosen for comparison. As shown in Table 3 a high binding affinity for the CCK2R with IC₅₀ values in the low nanomolar range could be confirmed for all tested peptidomimetics.

TABLE 3
Receptor binding of the inventive CCK2R targeting peptidomimetics
in comparison with pentagastrin, DOTA-MGSA, DOTA-MGSB and
DOTA-MGSC on A431-CCK2R cells as analyzed by displacement
with [¹²⁵I][3-iodo-Tyr¹², Leu¹⁵]gastrin-I.

| Tested CCK2R ligand | IC₅₀ [nM] |
|---|---|
| DOTA-MGSA | 0.21 ± 0.01 |
| DOTA-MGSB | 0.31 ± 0.02 |
| DOTA-MGSC | 0.16 ± 0.01 |
| pentagastrin | 0.37 ± 0.10 |

### Example 4: Peptidomimetics of the Present Invention Show Improved Biodistribution In Vivo

Biodistribution studies evaluating the tumour uptake of the radiolabelled inventive CCK2R targeting peptide analogues were performed in xenografted nude mice. All animal experiments were conducted with approval of the national authorities and carried out in compliance with the relevant European, national, and institutional regulations.

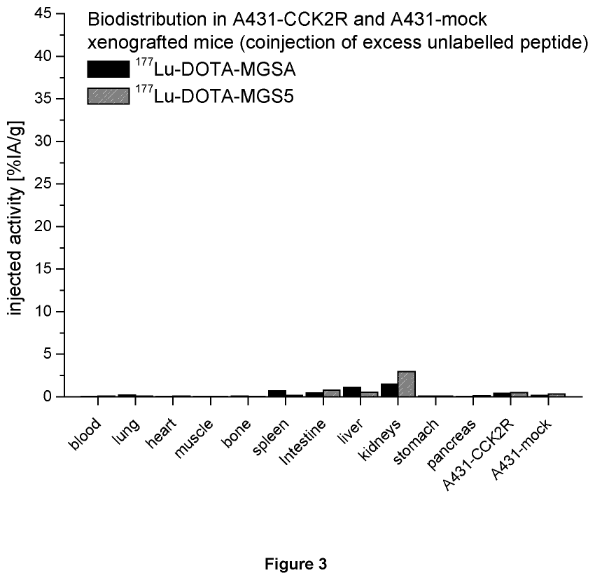

For the induction of tumour xenografts, A431-CCK2R and A431-mock cells were injected subcutaneously in athymic BALB/c nude mice (Charles River or Janvier Labs), respectively (2 million cells in 200 μL). When tumours had reached a size of approximately 0.2 ml, biodistribution studies were carried out. Groups of 3-5 mice were injected intravenously via a lateral tail vein with the peptidomimetics, labelled with ¹⁷⁷Lu (0.5-1 MBq and 0.02-0.03 nmol peptidomimetic) or ⁶⁸Ga (˜0.5 MBq and 0.02-0.03 nmol peptidomimetic). To check for receptor specificity blocking studies using co-injection of a 1000-fold molar excess of the unlabelled peptide (˜20 nmol) together with the administration of the ¹⁷⁷Lu-labelled peptide were performed (FIG. 3). After selected time points post-injection (p.i.) the animals were sacrificed by cervical dislocation, tumours and other tissues (blood, lung, heart, muscle, bone, spleen, intestine, liver, kidneys, stomach, pancreas) were removed, weighed, and their radioactivity measured in a gamma counter.

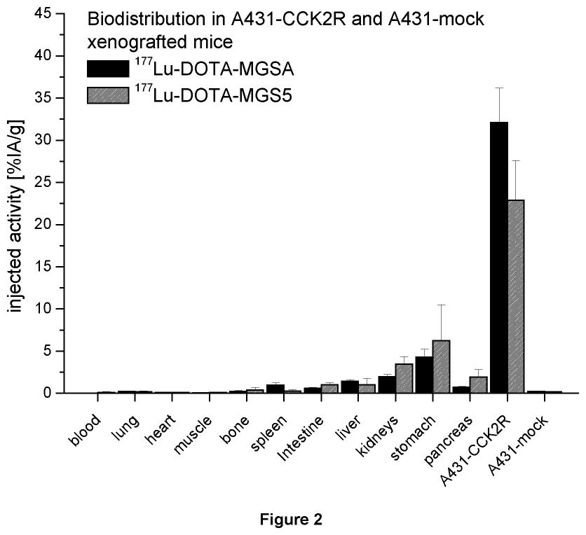

In a first study the biodistribution of ¹⁷⁷Lu-DOTA-MGSA and ¹⁷⁷Lu-DOTA-MGS5 at 4 h p.i. was compared (A431-CCK2R and A431-mock cells inoculated in the right and left flank of 7-week-old female athymic BALB/c nude mice; 5 animals per peptidomimetic). For one mouse in the ¹⁷⁷Lu-DOTA-MGSA group the bone sample was too small to measure the radioactivity, and one animal in the ¹⁷⁷Lu-DOTA-MGS5 group did not develop the A431-mock xenograft. Results were expressed as percentage of injected activity per gram tissue (% IA/g) and tumour to organ activity ratios were calculated from the activity measured in the dissected tissues (FIGS. 2 and 3).

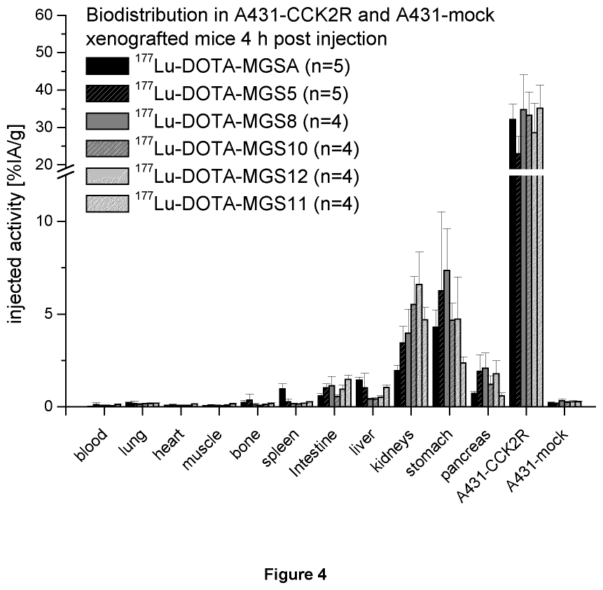

In FIG. 4 the results of the biodistribution studies at 4 h p.i. are summarized for the ¹⁷⁷Lu-labelled peptidomimetics of the present invention in comparison with ¹⁷⁷Lu-DOTA-MGS5 and other peptidomimetics of the prior art (Klingler M et al., J Nucl Med 2019, 60: 1010-1016; Klingler M et al., J Med Chem 2020, 63: 14668-14679). A favourable biodistribution profile with fast blood clearance, predominant renal excretion and low unspecific uptake in most tissues was observed for all radioligands. Differences were observed in the kidney uptake of the radiolabelled peptidomimetics with different amino acid sequence (see also FIG. 2). ¹⁷⁷Lu-DOTA-MGSA showed a lower kidney uptake of 2.0±0.3% IA/g. A higher kidney uptake of 3.5±0.9% IA/g was observed for ¹⁷⁷Lu-DOTA-MGS5. The peptidomimetic of the present invention further showed a high specific tumour uptake in A431-CCK2R xenografts with values of 32.1±4.1% IA/g for ¹⁷⁷Lu-DOTA-MGSA, which resulted to be significantly increased in comparison with, e.g., ¹⁷⁷Lu-DOTA-MGS5 (22.9±4.7% IA/g; p<0.01).

As shown in FIG. 4, the tumour uptake is comparable also to other ¹⁷⁷Lu-labelled peptidomimetics (¹⁷⁷Lu-DOTA-MGS8: 34.7±9.4% IA/g, ¹⁷⁷Lu-DOTA-MGS10: 33.3±6.3% IA/g, ¹⁷⁷Lu-DOTA-MGS12: 28.6±8.0% IA/g, ¹⁷⁷Lu-DOTA-MGS11: 35.1±6.3% IA/g) (Klingler M et al., J Med Chem 2020, 63: 14668-14679). Most strikingly, the tumour-to-kidney ratio of ¹⁷⁷Lu-DOTA-MGSA (16.7±2.7) was significantly increased when compared to ¹⁷⁷Lu-DOTA-MGS5 (7.0±2.2; p<0.001).

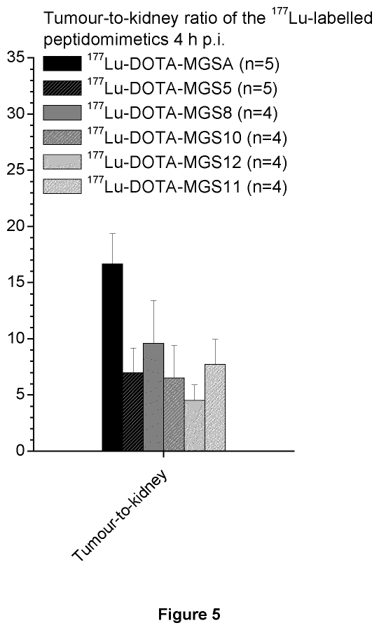

The tumour-to-kidney ratio of ¹⁷⁷Lu-DOTA-MGSA was increased also when compared to other ¹⁷⁷Lu-labelled peptidomimetics (¹⁷⁷Lu-DOTA-MGS8: 9.58±3.84, p<0.02; 177 Lu-DOTA-MGS10: 6.51±2.92, p<0.001; 177 Lu-DOTA-MGS12: 4.52±1.41, p<0.001; 177 Lu-DOTA-MGS11: 7.72±2.26, p<0.002; FIG. 5). The uptake in A431-mock tumour xenografts with values below <0.5% IA/g was very low and the uptake in A431-CCK2R xenografts was efficiently blocked to values of 0.39-0.54% IA/g by co-injection of an excess of unlabelled peptide, thus confirming the receptor-specific tumour uptake of the ¹⁷⁷Lu-labelled peptides studied (FIG. 3). Co-injection of an excess of unlabelled peptide further resulted in the abolishment of the receptor-specific uptake in stomach and pancreas, as well as reduction in kidney uptake.

In peptide receptor radionuclide radiotherapy usually multiple therapy cycles are administered to the patient in order to achieve a cumulative absorbed tumour radiation dose of 60 Gy allowing to reach an optimal therapeutic effect. A cumulative dose of <27 Gy for kidneys needs to be met to avoid renal toxicity (Konijnenberg M W et al., EJNMMI Res 201, 4, 47). The radiation dose delivered to the kidneys is a major factor limiting the total amount of radioactivity, which can be administered to patients. Consequently, the kidney uptake of the radiolabelled peptidomimetic influences the cumulative radiation dose, which can be achieved in the tumour lesions. Without being bound by theory, it is believed that the combination of high tumour uptake and improved tumour-to-kidney ratio of the peptidomimetics of the present invention allows administering a higher total amount of radioactivity in targeted radiotherapy reaching a higher absorbed dose in the tumour lesions while limiting the radiation dose delivered to healthy tissue (especially kidneys). No similar report exists on a comparable improvement in tumour-to-kidney ratio of a radiolabelled CCK2R targeting peptide analogue. The peptidomimetics of the present invention therefore display outstanding properties.

### Example 5: The Peptidomimetics of the Present Invention have Improved Targeting Properties for Peptide Receptor Radionuclide Therapy

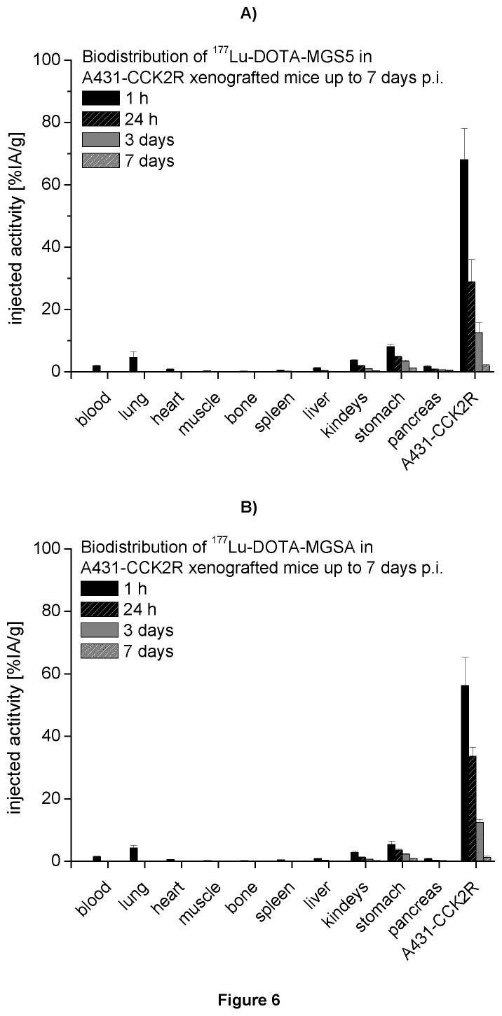

The biodistribution profile of ¹⁷⁷Lu-DOTA-MGSA and ¹⁷⁷Lu-DOTA-MGS5 over up to 7 days was studied in female athymic BALB/c nude mice, inoculated with A431-CCK2R cells (left flank) at an age of 6-8 weeks (5 animals per peptidomimetic and for each time point of 1 h, 24 h, 3 d and 7 d). When tumours had reached a size of approximately 0.1-0.2 ml, biodistribution studies were carried out. For both peptidomimetics an overall low non-specific tissue accumulation of radioactivity was observed. In FIG. 6, the biodistribution profile for the different time points of both peptidomimetics is displayed.

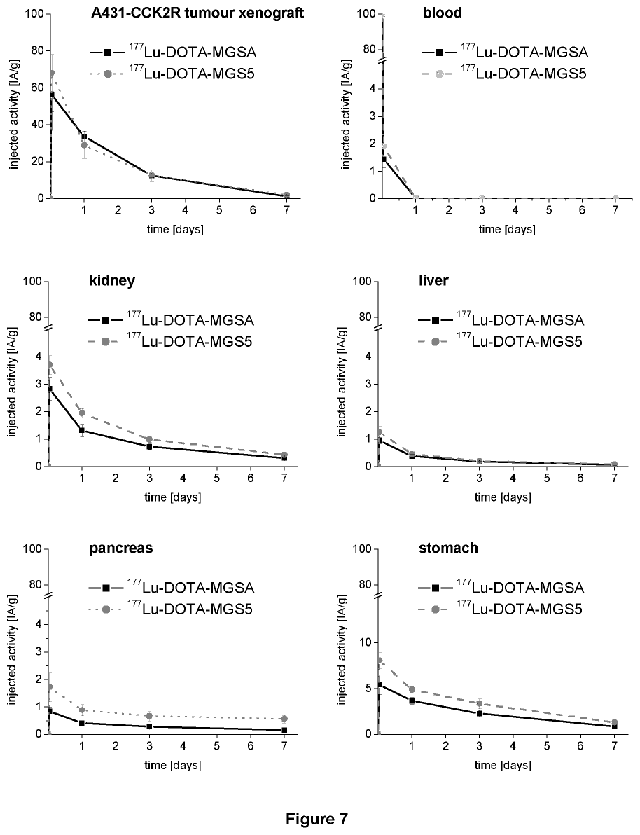

When considering the biodistribution over the 7 days, a significant difference was observed in some of the tissues. As shown in FIG. 7, ¹⁷⁷Lu-DOTA-MGSA showed significantly lower uptake levels in tissues with physiological CCK2R expression, pancreas (p<0.05 for all time points) and stomach (p<0.05 for 1 h, 24 h and 7 days p.i.) when compared to ¹⁷⁷Lu-DOTA-MGS5. Also the accumulation of radioactivity in excretory organs such as kidneys (p<0.05 for all time points) and liver (p<0.05 for 1 h and 7 days p.i.), was significantly lower. ¹⁷⁷Lu-DOTA-MGS5 also showed somewhat lower uptake levels in the blood, even though this difference was not significant. Both peptidomimetics showed a comparable tumour uptake over time, with values declining from 56.3±9.1% IA/g at 1 h p.i. to 1.2±0.5% IA/g at 7 d p.i. for ¹⁷⁷Lu-DOTA-MGSA and from 68.1±10.0% IA/g to 2.0±0.5% IA/g for ¹⁷⁷Lu-DOTA-MGS5. Without being bound by theory, it is believed that the combination of high tumour uptake and reduced retention of activity in off target tissue of the peptidomimetics of the present invention allows administering a higher total amount of radioactivity in targeted radiotherapy reaching a higher absorbed dose in the tumour lesions while limiting the radiation dose delivered to healthy tissue. The peptidomimetics of the present invention therefore display outstanding properties for therapy.

### Example 6: The Peptidomimetics of the Present Invention have Improved Targeting Properties for High Sensitivity Imaging

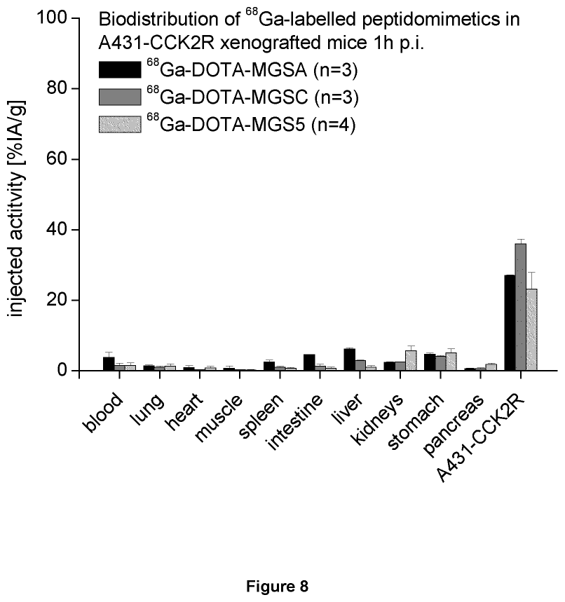

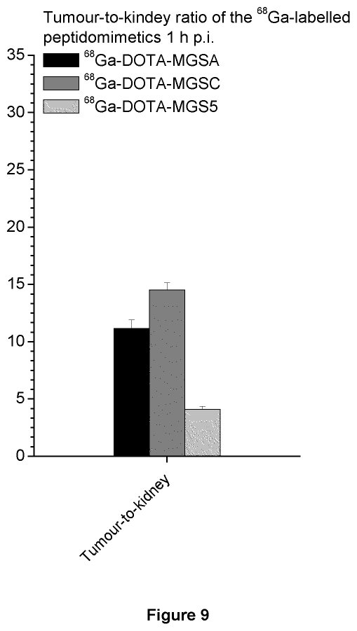

In further biodistribution studies the tumour uptake and imaging properties of different ⁶⁸Ga-labelled pepitdiomimetics was evaluated. For this purpose female athymic BALB/c nude mice (Janvier Labs, Le Genest Saint Isle, France) were inoculated with A431-CCK2R cells (left flank) at an age of 8-10 weeks. After a period of approximately 1 week, when tumours had reached a size of 0.1-0.2 ml, the animals were injected intravenously via a lateral tail vein with the peptidomimetics, labelled with ⁶⁸Ga (˜0.5 MBq and 0.02-0.03 nmol peptidomimetic). ⁶⁸Ga-DOTA-MGSA and ⁶⁸Ga-DOTA-MGSC both showed a favourable biodistribution profile with fast blood clearance, predominant renal excretion and low unspecific uptake in most tissues (see FIG. 8). A low kidney uptake was also confirmed for the ⁶⁸Ga-labelled peptidomimetics with values of 2.4±0.2% IA/g for ⁶⁸Ga-DOTA-MGSA and 2.5±0.1% IA/g ⁶⁸Ga-DOTA-MGSC. A much higher kidney uptake of 5.71±1.38% IA/g was previously reported for ⁶⁸Ga-DOTA-MGS5 (Klingler M et al., J Nucl Med 2019, 60: 1010-1016). The peptidomimetics of the present invention further showed a high specific tumour uptake in A431-CCK2R xenografts with values of 27.0±0.2% IA/g for ⁶⁸Ga-DOTA-MGSA and of 36.0±1.4% IA/g for ⁶⁸Ga-DOTA-MGSC. A tumour uptake of 23.3±4.7% IA/g was previously reported for ⁶⁸Ga-DOTA-MGS5 (Klingler M et al., J Nucl Med 2019, 60: 1010-1016). Especially the tumour uptake of ⁶⁸Ga-DOTA-MGSC was significantly increased when compared to ⁶⁸Ga-DOTA-MGSA (p<0.001) and ⁶⁸Ga-DOTA-MGS5 (p<0.01). When compared to ⁶⁸Ga-DOTA-MGS5 (4.1±0.3), the tumour-to-kidney ratio of ⁶⁸Ga-DOTA-MGSA (11.2±0.8; p<0.0001) and ⁶⁸Ga-DOTA-MGSC (14.5±0.6; p<0.000001) was significantly increased. The tumour-to-kidney of ⁶⁸Ga-DOTA-MGSC was also improved when compared to ⁶⁸Ga-DOTA-MGSA (p<0.005) (see FIG. 9).

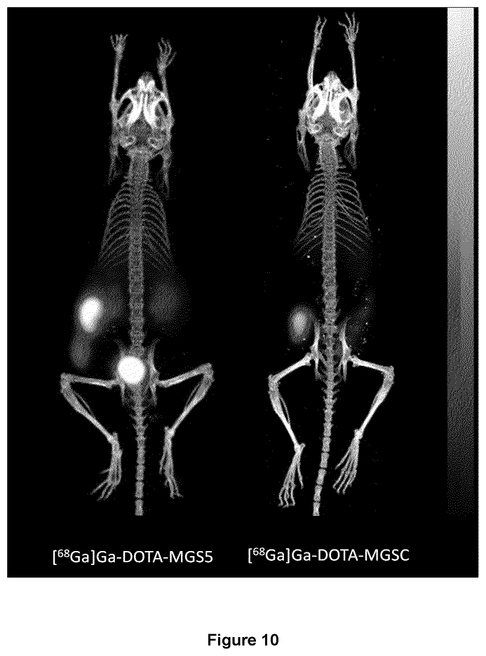

Small animal PET/CT imaging using a dedicated small animal μPET/CT scanner (Siemens Inveon PET/CT system) at an injected activity of ˜5 MBq (corresponding to 0.3 nmol peptide) confirmed the improved targeting properties of ⁶⁸Ga-DOTA-MGSC over ⁶⁸Ga-DOTA-MGS5, with best tumour-to-off target ratio allowing for a clear delineation of the tumour xenografts (see FIG. 10). Without being bound by theory, it is believed that the combination of high tumour uptake and reduced retention of activity in off target tissue of the peptidomimetics of the present invention allows for improved tumour detection in diagnostic applications. The peptidomimetics of the present invention therefore display outstanding properties for imaging.

### Example 7: The Peptidomimetics of the Present Invention has Increased Stability In Vivo

To further characterise the stability of the radiolabelled peptidomimetic in vivo, metabolic studies were carried out in 5-6-week-old female BALB/c mice (Charles River, Sulzfeld, Germany) injected intravenously with ¹⁷⁷Lu-labelled and ⁶⁸Ga-labelled peptidomimetics. All animal experiments were conducted in compliance with the Austrian animal protection laws and with the approval of the Austrian Ministry of Science. To allow monitoring of the metabolites by radio-HPLC, mice were injected with a higher amount of radioactivity (20-40 MBq ¹⁷⁷Lu, corresponding to ˜1 nmol total peptide, or ˜10 MBq ⁶⁸Ga, corresponding to ˜1 nmol total peptide) through a lateral tail vein and euthanized by cervical dislocation at selected time points p.i. (10 min for gallium-68 and 30 min for lutetium-177). A sample of blood was collected and the degradation was assessed by radio-HPLC. For this purpose blood samples were precipitated with ACN, centrifuged at 2000 g for 2 min and diluted with water (1:1/v:v) prior to HPLC analysis using a Dionex chromatography system including radiodetection and UV detection equipped with a Phenomenex Jupiter Proteo C12 column (90 Å, 4 μm, 250×4.6 mm) column using a water/acetonitrile/0.1% TFA gradient system.

The radiolabelled peptidomimetic of the present invention showed a very high stability against enzymatic degradation in vivo. The percentage of intact ¹⁷⁷Lu-DOTA-MGSA present in the blood at 30 min p.i. was 84.4% and compares with other ¹⁷⁷Lu-labelled peptidomimetics of the prior art (¹⁷⁷Lu-DOTA-MGS5: 77.0%, ¹⁷⁷Lu-DOTA-MGS8: 56.8%, ¹⁷⁷Lu-DOTA-MGS10: 86.1%, ¹⁷⁷Lu-DOTA-MGS12: 73.3%). At the time point of 10 min p.i. 92.8% of ¹⁷⁷Lu-DOTA-MGSA was intact and only 85.9% of ¹⁷⁷Lu-DOTA-MGS5. Also for ⁶⁸Ga-labelled DOTA-MGSA and DOTA-MGSB a high stability in vivo could be observed at 10 min p.i. with 95.1% and 94.7% intact radiopeptide, respectively. For ⁶⁸Ga-DOTA-MGSC 59.7% intact radiopeptide was observed for the same time point. The stability in vivo resulted to be also considerably increased when compared to ¹⁷⁷Lu-labelled PP-F11 and PP-F11 N.
PP-F11: DOTA-DGlu-DGlu-DGlu-DGlu-DGlu-DGlu-Ala-Tyr-Gly-Trp-Met-Asp-Phe-NH₂
PP-F11N: DOTA-DGlu-DGlu-DGlu-DGlu-DGlu-DGlu-Ala-Tyr-Gly-Trp-Nle-Asp-Phe-NH₂

All bonds (“—”) in PP-F11 and PP-F11N are amide bonds and all amino acids whose enantiomeric form is not expressly indicated are in the L-form.

These two peptide derivatives are derived from MG0 by substitution of the penta-Glu sequence with five D-glutamic acid residues and additional substitution of Met with Nle in PP-F11N. The two peptide conjugates were developed with the aim to improve the metabolic stability and pharmacokinetics and were first described in 2012 (Kroselj M et al., Eur J Nucl Med Mol Imaging 2012, 39: S533-S534 and WO 2015/067473 A1). For the same time point at 30 min p.i. ¹⁷⁷Lu-PP-F11 and ¹⁷⁷Lu-PP-F11N showed values of 5.5 and 12.7% intact radioligand present in the blood, respectively, and resulted to be almost completely degraded. In previous metabolic stability studies investigating the presence of intact radiopeptide in the blood and urine of different ¹⁷⁷Lu-labelled MG analogues, including also ¹⁷⁷Lu-PP-F-11, 10 min after intravenous injection into BALB/c mice no intact peptide could be found in urine and blood in vivo (Ocak M et al, Eur J Nucl Med Mol Imaging 2011, 38:1426-1435).

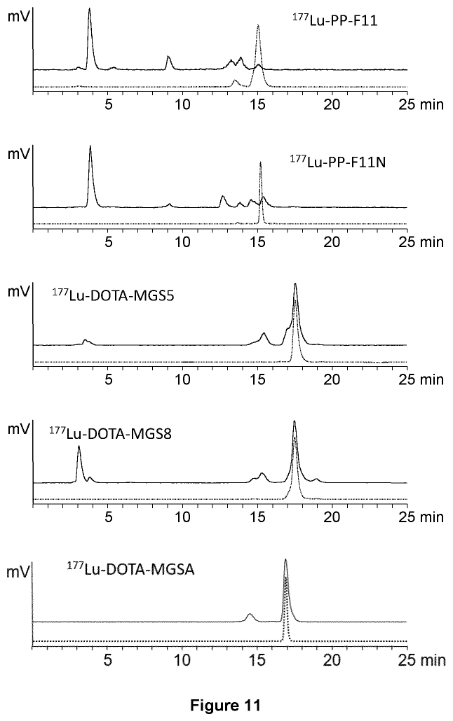

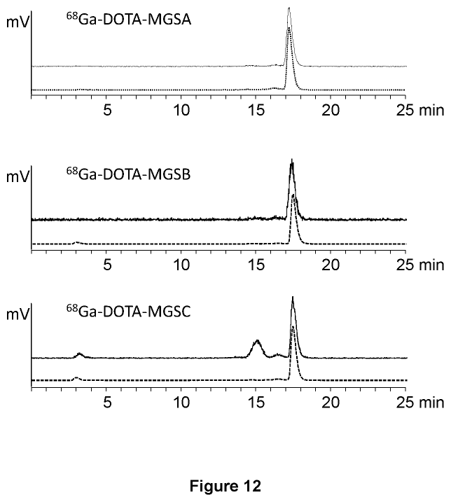

In FIG. 11, exemplary radiochromatograms of the blood samples obtained at 30 min p.i. (solid line) and of the radiochemical purity after radiolabelling (dotted line) are presented for ¹⁷⁷Lu-PP-F11 and ¹⁷⁷Lu-PP-F11N as well as ¹⁷⁷Lu-DOTA-MGS5, ¹⁷⁷Lu-DOTA-MGS8 and ¹⁷⁷Lu-DOTA-MGSA. Additional exemplary radiochromatograms of the blood samples after 10 min p.i. for ⁶⁸Ga-DOTA-MGSA, ⁶⁸Ga-DOTA-MGSB and ⁶⁸Ga-DOTA-MGSC are shown in FIG. 12.

The peptidomimetics of the present invention therefore displays a much higher stability against enzymatic degradation, as compared, for example, with PP-F11 and PP-F11N of the prior art. Surprisingly, the combination of substitutions in different positions, as according to the present invention, allows efficient stabilization of the peptidomimetic.

Without being bound by any particular theory, it is currently believed that improved in vivo stability may contribute to the improved tumour uptake and retention. The extremely high tumour uptake and tumour retention and most strikingly, the very favourable tumour-to-back-ground activity ratios, especially for kidneys, render the present peptidomimetic particularly useful for diagnostic and therapeutic uses in CCK2R relevant diseases, such as cancer.

1. A peptidomimetic with the following structure:
X-Linker-βAla-Trp-(NMe)Nle-Asp-1Nal,
wherein X is a chelator comprising a radionuclide or a prosthetic group comprising a radionuclide.

2. The peptidomimetic of claim 1, wherein the chelator or the prosthetic group is bound to a radionuclide selected from the group consisting of:
²²⁵Ac, ²¹²Bi, ²¹³Bi, ⁶²Cu, ⁶⁴Cu, ⁶⁷Cu, ⁶⁹Cu, ⁶⁶Ga, ⁶⁷Ga, ⁶⁸Ga, ¹¹¹In, ¹¹3 min, ¹⁷⁷Lu, ¹⁸⁶Re, ¹⁸⁸Re, ⁴³Sc, ⁴⁴Sc, ⁴⁷Sc, ¹⁵⁵Tb, ¹⁶¹Tb, ^{99m}Tc, ⁸⁶Y, ⁹⁰Y, ¹⁶⁹Yb, ¹⁷⁵Yb, ⁵²Fe, ¹⁶⁹Er, ⁷²As, ⁹⁷Ru, ²⁰³Pb, ²¹²Pb, ⁵¹Cr, ^{52m}Mn, ⁸⁹Zr, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ¹⁴²Pr, ¹⁴³Pr, ¹⁹⁸Au, ¹⁹⁹Au, ¹²³I, ¹²⁴I, ¹²⁵I, Al¹⁸F and ¹⁸F.

3. The peptidomimetic of claim 1, wherein the chelator is 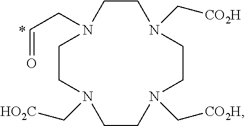 [SMILES: O=CCN1CCN(CC(=O)O)CCN(CC(=O)O)CCN(CC(=O)O)CC1]
wherein the asterisk indicates the position where the chelator is directly bound to the Linker.

4. The peptidomimetic of claim 1, wherein the radionuclide is chelated by the chelator.

5. The peptidomimetic of claim 1, wherein the radionuclide is bound to the prosthetic group by a covalent bond.

6. The peptidomimetic of claim 5, wherein the radionuclide is a radionuclide of a halogen.

7. The peptidomimetic of claim 1, wherein the peptidomimetic is DOTA-Linker-βAla-Trp-(NMe)Nle-Asp-1Nal.

8. The peptidomimetic of any one of claims 1 to 7, wherein the Linker is selected from the group consisting of GABA-GABA, GABOB-GABOB or γDGlu-γDGlu.

9. The peptidomimetic of claim 8, wherein the peptidomimetic is DOTA-GABOB-GABOB-βAla-Trp-(NMe)Nle-Asp-1Nal.

10. The peptidomimetic of claim 9, wherein the peptidomimetic comprises a radionuclide that is chelated by DOTA.

11. The peptidomimetic of claim 10, wherein the radionuclide is selected from the group consisting of ²²⁵Ac, ²¹²Bi, ²¹³Bi, ⁶²Cu, ⁶⁴Cu, ⁶⁷Cu, ⁶⁹Cu, ⁶⁶Ga, ⁶⁷Ga, ⁶⁸Ga, ¹¹¹In, ^{113m}In, ¹⁷⁷Lu, ¹⁸⁶Re, ¹⁸⁸Re, ⁴³Sc, ⁴⁴Sc, ⁴⁷Sc, ¹⁵⁵Tb, ¹⁶¹Tb, ^{99m}TD, ⁸⁶Y, ⁹⁰Y, ¹⁶⁹Yb, Al¹⁸F and ¹⁷⁵Yb.

12. The peptidomimetic of claim 11, wherein the radionuclide is ⁹⁰Y, ¹¹¹In, ⁶⁸Ga, ²²⁵Ac or ¹⁷⁷Lu.

13. The peptidomimetic of claim 12, wherein the peptidomimetic is ⁶⁸Ga-DOTA-GABOB-GABOB-βAla-Trp-(NMe)Nle-Asp-1Nal-NH₂.

14. The peptidomimetic of claim 12, wherein the peptidomimetic is ¹⁷⁷Lu-DOTA-GABOB-GABOB-βAla-Trp-(NMe)Nle-Asp-1Nal-NH₂.

15. A method of producing the peptidomimetic of any one of claims 1 to 14, comprising synthesizing the peptidomimetic.

16. A pharmaceutical composition comprising the peptidomimetic of any one of claims 1 to 14 and a pharmaceutically acceptable carrier.

17. Use of the peptidomimetic of any one of claims 1 to 14 or the pharmaceutical composition of claim 16 for imaging a tumor.

18. A method of imaging cells, wherein the method comprises the steps of
a) contacting the cells with the peptidomimetic of any one of claims 1 to 14 or the pharmaceutical composition of claim 16, thereby bringing the radionuclide in contact with the cells, and
b) visualizing the radionuclide that is in contact with the cells.

19. The method of claim 18, wherein contacting comprises administering the peptidomimetic to a patient.

20. The method of claim 18 or 19, wherein the cells express CCK2R on the surface of the cells.

21. The method of any one of claims 18 to 20, wherein the cells are cancer cells.

22. The method of any one of claims 18 to 21, wherein the peptidomimetic is ⁶⁸Ga-DOTA-GABOB-GABOB-βAla-Trp-(NMe)Nle-Asp-1Nal-NH₂.

23. The peptidomimetic of any one of claims 1 to 14 or the pharmaceutical composition of claim 16 for use in therapy.

24. The peptidomimetic for use according to claim 23, wherein the peptidomimetic is ⁶⁸Ga-DOTA-GABOB-GABOB-βAla-Trp-(NMe)Nle-Asp-1Nal-NH₂ or ¹⁷⁷Lu-DOTA-GABOB-GABOB-βAla-Trp-(NMe)Nle-Asp-1Nal-NH₂.

25. The peptidomimetic of any one of claims 1 to 14 or the pharmaceutical composition of claim 16 for use in the treatment of a cancer.

26. The peptidomimetic for use according to claim 25, wherein the cancer expresses CCK2R on the surface of cancer cells.

27. The peptidomimetic for use according to claim 25 or 26, wherein the peptidomimetic is ⁶⁸Ga-DOTA-GABOB-GABOB-βAla-Trp-(NMe)Nle-Asp-1Nal-NH₂ or ¹⁷⁷Lu-DOTA-GABOB-GABOB-βAla-Trp-(NMe)Nle-Asp-1Nal-NH₂.

28. The peptidomimetic of any one of claims 1 to 14 or the pharmaceutical composition of claim 16 for use in diagnosing a cancer.

29. The peptidomimetic for use according to claim 28, wherein the cancer expresses CCK2R on the surface of cancer cells.

30. The peptidomimetic for use according to claim 28 or 29, wherein the peptidomimetic is ⁶⁸Ga-DOTA-GABOB-GABOB-βAla-Trp-(NMe)Nle-Asp-1Nal-NH₂.

31. A method of treating a patient suffering from a disease, the method comprising administering to the patient the peptidomimetic of any one of claims 1 to 14 or the pharmaceutical composition of claim 16.

32. The method of claim 31, wherein the disease is a cancer.

33. The method of claim 32, wherein the cancer expresses CCK2R on the surface of cancer cells.

34. The method of any one of claims 31 to 33, wherein the peptidomimetic is ⁶⁸Ga-DOTA-GABOB-GABOB-βAla-Trp-(NMe)Nle-Asp-1Nal-NH₂ or ¹⁷⁷Lu-DOTA-GABOB-GABOB-βAla-Trp-(NMe)Nle-Asp-1Nal-NH₂.

35. A method of diagnosing cancer in a patient, wherein the method comprises the steps of
a) contacting a cancer cell of the patient with the peptidomimetic of any one of claims 1 to 14 or the pharmaceutical composition of claim 16, thereby bringing the radionuclide in contact with the cancer cell, and
b) visualizing the radionuclide that is in contact with the cancer cell.

36. The method of claim 35, wherein the cancer expresses CCK2R on the surface of cancer cells.

37. The method of claim 35 or 36, wherein the peptidomimetic is ⁶⁸Ga-DOTA-GABOB-GABOB-βAla-Trp-(NMe)Nle-Asp-1Nal-NH₂ or ¹⁷⁷Lu-DOTA-GABOB-GABOB-βAla-Trp-(NMe)Nle-Asp-1Nal-NH₂.

38. Use of the peptidomimetic of any one of claims 1 to 14 or the pharmaceutical composition of claim 16 for diagnosing cancer.

39. Use of the peptidomimetic of any one of claims 1 to 14 or the pharmaceutical composition of claim 16 for treating cancer.

40. Use of the peptidomimetic of any one of claims 1 to 14 or the pharmaceutical composition of claim 16 for distinguishing a cancer cell from a healthy cell.

41. The use of any one of claims 38 to 40, wherein the cancer expresses CCK2R on the surface of cancer cells.

42. The use of any one of claims 38 to 41, wherein the peptidomimetic is ⁶⁸Ga-DOTA-GABOB-GABOB-βAla-Trp-(NMe)Nle-Asp-1Nal-NH₂ or ¹⁷⁷Lu-DOTA-GABOB-GABOB-βAla-Trp-(NMe)Nle-Asp-1Nal-NH₂.


SEQUENCE LISTING (7 sequences)

SEQ ID NO: 1 - protein, 8 aa, Artificial Sequence [sequence omitted]

SEQ ID NO: 2 - protein, 13 aa, Artificial Sequence
  artificial peptide
  amidated at the C-terminus
LEEEEEAYGWMDF

SEQ ID NO: 3 - protein, 8 aa, Artificial Sequence
  MOD_RES 1: DOTA (1,4,7,10-tetraazacyclododecan-1,4,7,10- tetra acetic acid) modified D-Glu chelated with 111 Indium
  111In-DOTA-MGS1
  MOD_RES 8: Xaa = 1-naphtylalanine (1Nal) amidated at the C-terminus
XAYGWMDX

SEQ ID NO: 4 - protein, 8 aa, Artificial Sequence
  MOD_RES 1: DOTA (1,4,7,10-tetraazacyclododecan-1,4,7,10- tetra acetic acid) modified D-Glu chelated with 111 Indium
  111In-DOTA-MGS4
  MOD_RES 6: Xaa = Nle -CONCH3- pseudopeptide bonded to Trp
  MOD_RES 8: Xaa = Phenylalanine -CONCH3- pseudopeptide bonded, amidated at the C-terminus
XAYGWXDX

SEQ ID NO: 5 - protein, 8 aa, Artificial Sequence
  MOD_RES 1: DOTA (1,4,7,10-tetraazacyclododecan-1,4,7,10- tetra acetic acid) modified D-Glu
  DOTA-MGS5
  MOD_RES 6: Xaa = Nle -CONCH3- pseudopeptide bonded to Trp
  MOD_RES 8: Xaa = 1-naphtylalanine (1Nal) amidated at the C-terminus
XAYGWXDX

SEQ ID NO: 6 - protein, 5 aa, Artificial Sequence
  MOD_RES 1: beta-Alanine optionally having attached DOTA by a linker, linker may be 2x GABOB, 2x GABA, 1x DGlu or 2x gamma-DGlu
  peptidomimetic binding to CCK2R
  MOD_RES 3: Xaa = Nle -CONCH3- pseudopeptide bonded to Trp
  MOD_RES 5: Xaa = 1Nal
XWXDX

SEQ ID NO: 7 - protein, 5 aa, Artificial Sequence
  MOD_RES 1: beta-Alanine having attached DOTA by linker, linker may be 2x GABOB, 2x GABA, 1x DGlu or 2x gamma-DGlu, DOTA being chelated with either 68Ga or 177Lu
  radionuclide chelated peptidomimetic binding to CCK2R
  MOD_RES 3: Xaa = Nle -CONCH3- pseudopeptide bonded to Trp
  MOD_RES 5: Xaa = 1Nal, amidated at C-terminus
XWXDX